(19) United States
(12) Patent Application Publication
(10) Pub. No.: US 2010/0021447 A1
(43) Pub. Date: Jan. 28, 2010

(54) Medicament for Treating Problems Relating to Fertility and Pregnancy, and Autoimmune Diseases, and for Inducing an Immunological Tolerance in Transplant Patients, and Method for Producing Said Medicament

(75) Inventors: Henry Alexander, Leipzig (DE); Gerolf Zimmermann, Leipzig (DE)

Correspondence Address: GUDRUN E. HUCKETT DRAUDT, SCHUBERTSTR. 15A, WUPPERTAL, 42289, DE

(73) Assignee: UNIVERSITÄT LEIPZIG, Leipzig (DE)

(21) Appl. No.: 12/094,275

(22) PCT Filed: Nov. 21, 2006
(86) PCT No.: PCT/DE06/02089

§ 371 Date: Jan. 21, 2009

(30) Foreign Application Priority Data
Nov. 22, 2005 (DE) 10 2005 056 832.7

(51) Int. Cl. A61K 35/12; A61K 38/16

(52) U.S. Cl. 424/93.71; 514/12

(57) ABSTRACT

A medicament for treating pregnancy disorders or for inducing an immunological tolerance in patients with autoimmune diseases or transplantation processes, contains at least one each of a) a precursor hCG β subunit of the human choriongonadotropine (hCG) selected from hCG β6 according to SEQ ID NO 1 or SEQ ID NO 2 and hCG β7 according to SEQ ID NO 5 or a mature hCG β subunit selected from hCG β6 according to SEQ ID NO 3 or SEQ ID NO 4 and hCG β7 according to SEQ ID NO 6 or glycolised fragments of these sequences; and b) a precursor α subunit of hCG according to SEQ ID NO 9 or the mature α subunit of hCG according to SEQ ID NO 10 or glycolysed fragments of these sequences, wherein the β subunits and the α subunits are preferably used in equimolar quantities.

The invention concerns a medicament for treating problems relating to fertility and pregnancy, autoimmune diseases, and for inducing an immunological tolerance in transplant patients for use in medicine, especially in gynecology and transplant medicine, as well as a method for its preparation.

In gynecology premature birth presents a great medical problem. In case of premature birth the pregnancy ends before the 37th week of gestation (normal duration of gestation: 40 weeks). In Germany the premature birth rate is approximately 6 to 7%. Despite great efforts, it has not been possible to lower the premature birth rate in the past decades. Approximately two-thirds of perinatal deaths of newborns are the result of premature birth. “Preemies” have a weight of 500 to 2,500 g. The neonatal care of the preemies often takes several months and is a very cost-intensive field of pediatrics. Despite medical intensive care approximately 70% of premature birth babies experience long-term damage (neurological damage, bodily and mental developmental problems or retardation, visual or hearing impairment).

In the past there has been no causal therapy for late pregnancy problems or premature birth. In early pregnancy disorders (fertility problems, implantation problems, early pregnancy losses, imminent and habitual abortion) progesterone is prescribed for stabilization. Partially, hCG is also administered up to the 10th week of pregnancy. The hCG that has been administered up to now is trophoblastic hCG.

Late pregnancy disorders (premature birth, preeclampsia, growth retardation) are currently treated symptomatically with progesterone, magnesium, β-sympathicomimetic drugs or anti-hypertonic agents.

For years, immunosuppressive agents have been administered in patients with organ transplants and autoimmune diseases. In these cases, these medicaments must be administered permanently. However, they cause considerable side effects that are responsible for increased morbidity and mortality. Therefore, for years it has been attempted to provide new methods and medicaments for the treatment of patients with organ transplants and autoimmune diseases.

For immune tolerance also the Fas ligand is of importance (Fandrich F., Lin X., Kloppel G., and Kremer B., 1998; Fandrich F., Lin X., Zhu X., Parwaresch R., Kremer B., and Henne-Bruns D., 1998).

The hormones human choriongonadotropine (hCG), LH (luteinizing hormone), FSH (follicle stimulating hormone) and TSH (thyroid stimulating hormone) form a family of glycoprotein hormones. They are comprised of non-covalently bonded heterodimers of an α subunit and a β-subunit. The α-subunit is identical in all four hormones and the β-subunits differ from one another and define the endocrine function of the heterodimers (Pierce et al., 1981). The β-subunit of the choriongonadotropine differs from the other β-subunits of the glycoprotein hormones mainly in that it is extended at the C-terminal by 23 amino acids—the so-called C-terminal peptide (CTP). The β-subunit of the hCG has two asparagine-N-glycosidic side chains, the C-terminal peptide (CTP) part (amino acids 122 to 145) has four additional serine-O-glycosidic oligosaccharide side chains.

The α subunit of LH, FSH, TSH, and of human choriongonadotropine (αCG) is coded by a gene that is localized on chromosome 6 (chromosome 6q21.1-q23) while the β-subunit of the human choriongonadotropine (βhCG) is coded by the six homolog genes hCG β1, β2, β3, β5, β7, and β8 that are localized as a gene cluster on chromosome 19 (chromosome 19q13.3) adjacent LH β4 (Jameson et al., 1993). The βhCG gene β6 is most likely an allele of β7 with differences in non-translating nucleotide sequence of the promoter gene (exon 1) and the translating sequence (exon 2) of the βhCG subunit.

During pregnancy in the trophoblast of the early embryo (beginning at the 6th to 12th day after conception) and later in the syncytiotrophoblast of the placenta large quantities of hCG heterodimer and free α-CG and the βhCG subunits are generated and secreted into the blood. This trophoblastic tissue expresses exclusively the β-hCG subunits hCG β5, β8, and β3. These βhCG subunits are therefore referred to as trophoblastic βhCG (tβhCG) or type-II-βhCG. This trophoblastic hCG binds to the corpus luteum that in this way is induced to produce and secrete more progesterone that is required for maintaining the pregnancy.

The trophoblastic hCG acts like LH on a common membrane-bonded G-protein-coupled receptor. It can be detected in the epithelium, endothelium, and the stroma cells of the endometrium and other organs, in lymphocytes and macrophages (Reshef et al., 1990; Licht et al., 1993; Lin et al., 1996; Zhang et al., 2003; Licht et al., 2003). In addition, the possibly trophoblastic βhCG also acts through signal pathways that are not receptor-translated (Cruz et al., 1987).

But in some non-trophoblastic tissues hCG heterodimers or free αCG and βhCG subunits are also expressed in minimal quantities (Rothman et al., 1992; Dirnhofer et al., 1996; Lei et al., 1993; Yokotani et al., 1997; Berger et al., 1994). Non-trophoblastic tissue, e.g., mamma, lung, prostate, bladder, and colon, express exclusively the βhCG subunits hCG β7/β6. In the endometrial and decidual epithelium of the uterus non-trophoblastic hCG is formed also (Alexander et al., 1998b; Wolkersdorfer et al., 1998; Zimmermann et al., 2003). The βhCG subunits β7 or the β7 allele β6 are therefore also referred to as non-trophoblastic or epithelial βhCG or type I βhCG (Bellet et al., 1997). The function of the non-trophoblastic hCG however has hardly been elucidated.

Object of the invention is therefore to provide an agent for treatment of pregnancy disorders, in particular for treatment of fertility problems, implantation problems, early pregnancy losses, imminent and habitual abortion as well as premature birth, growth retardation, and preeclampsia.

Object of the invention is also to provide an agent for treatment of autoimmune diseases and for induction of immune tolerance in transplant patients.

According to the invention this object is solved by a medicament in particular for treatment of pregnancy disorders that comprises a precursor-hCG β subunit selected from hCG β6 according to SEQ ID NO 1 or alternatively SEQ ID NO 2, from hCG β7 according to SEQ ID NO 5 or a mature hCG β subunit selected from hCG β6 according to SEQ ID NO 3 or alternatively SEQ ID NO 4, from hCG β7 according to SEQ ID NO 6 or fragments thereof.

The invention also encompasses the use of a precursor-hCG β subunit selected from hCG β6 according to SEQ ID NO 1 or SEQ ID NO 2 and hCG β7 according to SEQ ID NO 5 or a mature hCG β-subunit selected from hCG β6 according to SEQ ID NO 3 or SEQ ID NO 4, hCG β7 according to SEQ ID NO 6 or glycan-linked oligopeptide fragments thereof for treatment of pregnancy disorders.

The amino acid sequences according to SEQ ID NO 1 or SEQ ID NO 2 represent the two possible forms of amino acid sequences of the precursor of the endometrial or decidual hCG β6 subunit. The precursor of the hCG β6 subunit is comprised of 165 amino acids (in SEQ ID NO 1 or SEQ ID NO 2 numbered from 1 to 165). The amino acid sequence of amino acids 1 to 20 is the signal peptide that is cleaved off in the Golgi apparatus. The specific mature form of the decidual hCG β6-subunit corresponds to the amino acid sequence of amino acid 21 to amino acid 165, i.e., an amino acid sequence according to SEQ ID NO 3 or SEQ ID NO 4.

The specific mature form of the endometrial or decidual hCG β6-subunit is comprised of 145 amino acids (in SEQ ID NO 3 or SEQ ID NO 4 numbered from 1 to 145). The amino acid sequence of hCG β6-subunit comprises in contrast to the amino acid sequence of the trophoblastic hCG β7 subunits β3, β5, and β8 at amino acid position 117 Ala instead of aspartate. At position 2 of hCG β6-subunit there is lysine (SEQ ID NO 3) or arginine (SEQ ID NO 4).

Preferred is the mature form of the endometrial or decidual hCG β6-subunit according to SEQ ID NO 3 or SEQ ID NO 4 and/or the precursor of the decidual hCG β6-subunit according to SEQ ID NO 1 or SEQ ID NO 2 in the medicament.

SEQ ID NO 5 is the precursor of the amino acid sequence of the endometrial or decidual hCG β7-subunit. The precursor of the hCG β7-subunit is comprised of 165 amino acids (numbered from 1 to 165 in SEQ ID NO 5). The amino acid sequence of amino acid 1 to 20 corresponds to the signal peptide that is cleaved off in the Golgi apparatus. The specific biologically mature form of the endometrial or decidual hCG β7-subunit corresponds to the amino acid sequence of amino acid 21 to amino acid 165, i.e., an amino acid sequence according to SEQ ID NO 6.

The mature form of the endometrial or decidual hCG β7-subunit is comprised of 145 amino acids (numbered 1 to 145 in SEQ ID NO 6). The amino acid sequence of hCG β7-subunit comprises in contrast to the amino acid sequence of the trophoblastic hCG β-subunits β3, β5, β8 at amino acid position 117 alanine instead of aspartate. At amino acid position 2 it contains arginine instead of lysine, at amino acid position 4 it contains methionine instead of proline.

Preferably, the mature form of the endometrial or decidual hCG β7-subunit according to SEQ ID NO 6 and/or the precursor of the decidual hCG β7-subunit according to SEQ ID NO 5 is contained in the medicament according to the invention.

In a preferred embodiment of the medicament the latter contains in addition to the endometrial β6 unit and/or β7 unit the trophoblastic subunit hCG β5, hCG β3, and hCG β8 according to SEQ ID NO 7 and/or SEQ ID NO 8.

SEQ ID NO 7 is the precursor of the amino acid sequence of the trophoblastic βhCG subunits β5, β3 and β38. The precursor of the trophoblastic βhCG subunit β5, β3, and β8 each are comprised of 165 amino acids (in SEQ ID NO 7 numbered 1 to 165). The amino acid sequence of amino acid 1 to 20 corresponds to the signal peptide that is cleaved off in the Golgi apparatus. The specific mature forms of the trophoblastic βhCG subunits β5, β3 and β8 correspond to the amino acid sequence of amino acid 21 to amino acid 165, i.e., an amino acid sequence according to SEQ ID NO 8.

The specific mature forms of the trophoblastic βhCG subunits β5, β3, and β8 are comprised of 145 amino acids (in SEQ ID NO 8 numbered 1 to 145). The amino acid sequence of the βhCG subunits β5, β3, and β8 contains in contrast to the amino acid sequence of the decidual βhCG subunits β6 and β7 at amino acid position 117 an aspartate instead of alanine. At amino acid position 2 it contains lysine, at amino acid position 4 it contains proline.

With the medicament according to the invention for the first time it is possible to carry out a causal therapy of pregnancy disorders. The loss of decidual hCG that is the cause for pregnancy disorders is substituted by the medicament according to the invention. At the same time, the administered hCG stimulates the formation of hCG in the decidua which, in turn, sedates the uterus muscles and improves blood flow for the placenta. In this way, a causal treatment of pregnancy disorders and premature onset of birth, meaning premature birth, is enabled.

In case of the hCG preparations that have been used in the past in gynecology, purified urinary hCG or gene-technologically recombinant-produced human choriongonadotropine is used. This hCG is comprised of the βhCG subunit β5, β8, β3 as well as the αCG subunit and has a main site of action at the yellow body (corpus luteum) of the ovaries. Since the corpus luteum in humans is essential only up to the 10th week of gestation, in accordance with the prior art an hCG therapy is carried out only up to 10th week of gestation.

Human decidual choriongonadotropine that is comprised of the hCG β-subunit β6 or β7 and the αCG-subunit supports also the corpus luteum but is mainly required for maintaining the immune tolerance of pregnancy. The induction, expression, and protein formation of βhCG gene β7 and/or β6 in the endometrial and the decidual gland epithelium enhances fertility and pregnancy. In this connection, the effect is possible by means of several mechanisms.

Pregnancy itself is an immunological paradox. The embryo or fetus is a so-called “semi allotransplant” where one half is comprised of the maternal and the other half of the paternal genes and therefore is one half “foreign”. The embryo must therefore be tolerated immunologically for 38 weeks up to maturity. This is a complex and multifaceted process and hardly anything is presently known about its mechanisms.

The uterus is an immune-privileged site. In this connection, the endometrial hCG (β6/β7 hCG and αCG) represents the main factor for this biological peculiarity that enables a successful pregnancy.

hCG acts by immunosuppressive action on the uterus. In this way, the decidua that envelopes the embryo and by means of the fetal membrane also releases hCG into the amniotic fluid acts like a protective shield. The hCG of the decidua (β6/β7 hCG and αhCG) is at the same time responsible for chemotactic attraction of mononuclear immune cells that on their part prevent a rejection reaction. Moreover, the decidual hCG improves the blood flow of the uterus and the placenta.

Only when the ability for hCG production and secretion by the decidua decreases at the end of gestation primarily as a result of the decreasing progesterone level, the immune tolerance of gestation is terminated also, the local immune protection is canceled, the protective mononuclear cells become apoptotic and the hCG-induced optimal blood flow of decidua and placenta is reduced. This causes hypoxia and necrosis of the decidua and thus the generation of prostaglandins as well as oxytocin and leads to birth. In patients experiencing premature birth this process is prematurely induced by a plurality of disturbances.

Therefore, in case of lack of decidual choriongonadotropine its substitution during the entire pregnancy up to the 37th week of pregnancy is required.

The medicament according to the invention serves for treatment of pregnancy disorders. Pregnancy disorders are to be understood as fertility disorders, implantation problems, early pregnancy losses, imminent and habitual abortion as well as premature birth, growth retardation and preeclampsia. In particular, pregnancy disorders are included that are caused by a lack of decidual hCG.

A fertility disorder relates to a disturbance that is characterized in that no pregnancy happens despite regular unprotected intercourse.

An implantation problem is present when the egg is fertilized but will not implant in the endometrium.

Early pregnancy losses are characterized in that an embryo has implanted in the endometrium but the embryo shortly thereafter will die off.

An imminent abortion is a so-called imminent miscarriage that is usually characterized by bleeding and abdominal pain. While on the other hand a habitual miscarriage tendency is present when a patient has already experienced a miscarriage three or multiple times in sequence.

A premature birth is present when the birth takes place between the 24th and 37th week of gestation, particularly when a life birth occurs even before the 24th week of gestation.

An intrauterine growth retardation means that the fetus for his age is too small in relation to the week of gestation. In this connection, the deviation of the estimated weight is below the normal value by two standard deviations. This deviation of the growth is determined by measuring the fetus by ultrasound and subsequent comparison with growth charts.

Preeclampsia is a hypertensive disease during pregnancy (pregnancy hypertension). It describes at the same time the presence of edema and protein secretion in the urine. In 20% of the cases the liver is involved also with increase of transaminases and of bilirubin.

With the agents according to the invention the treatment of autoimmune diseases is also made possible. Moreover, the agents according to the invention are suitable also for induction of immune tolerance in transplant patients.

The term immune tolerance refers to the lack of an immune reaction after administration of a certain antigen. The term autoimmune disease is a collective term for diseases whose cause relates to an excessive reaction of the immune system against the body's own tissue. In this connection, the immune system perceives the body's own tissue erroneously as a foreign body that must be attacked. In this way, severe systemic or local inflammation reactions occur that can damage the concerned organs.

hCG is an immune-suppressive substance. Therefore, an hCG therapy in the aforementioned way can suppress an immune reaction of the body against an allotransplant, i.e., an organ transplant from another individual, as well as suppress also an erroneous immune response against the body's own tissue in the context of autoimmune diseases.

Preferably, the medicament contains additionally the precursor of the αCG subunit of the human choriongonadotropine according to SEQ ID NO 9 or the mature αCG-subunit of the human choriongonadotropine according to SEQ ID NO 10 or glycan-linked oligopeptide fragments as parts of these sequences.

The SEQ ID NO 9 is the amino acid sequence of the precursor of the hCG α-subunit (J. C. Fiddes and H. M. Goodman, 1973). The precursor of the hCG α-subunit is comprised of 116 amino acids (here numbered from 1 to 116). The amino acid sequence of amino acid 1 to 24 corresponds to the signal peptide that is cleaved off in the Golgi apparatus. The specific mature form of the hCG α-subunit corresponds to the amino acid sequence of amino acid 25 to amino acid 116, i.e., the amino acid sequence according to SEQ ID NO 10.

The specific mature form of hCG α-subunit is comprised of 92 amino acids (numbered 1 to 94 in SEQ ID NO 10). Preferably, the mature hCG α-subunit according to SEQ ID NO 10 is contained in the medicament according to the invention.

When the medicament for treatment of pregnancy disorders, fertility disorders or autoimmune diseases and for induction of immunological tolerance contains, in addition to the hCG β-subunit, also the αCG subunit of the human choriongonadotropine according to SEQ ID NO 9 or SEQ ID NO 10 or glycan-linked oligopeptide fragments thereof, preferably equimolar quantities of βhCG subunits and αCG subunits are present. When the medicament is comprised, for example, of the hCG β-subunit β6 according to SEQ ID NO 1 and the α-subunit of the human choriongonadotropine according to SEQ ID NO 9, the medicament according to the invention contains preferably equimolar quantities of hCG β-subunit β6 according to SEQ ID NO 1 and of the α-subunit of the human choriongonadotropine according to SEQ ID NO 9.

When the medicament for treatment of pregnancy disorders contains different forms of the βhCG subunit such as βhCG β6 and/or β7, the medicament preferably contains an αCG subunit of the human choriongonadotropine according to SEQ ID NO 9 or SEQ ID NO 10 or fragments thereof for each βhCG subunit contained in the medicament or for each fragment of hCGβ subunits contained in the medicament. When the medicament contains, for example, the hCG β-subunit β6 according to SEQ ID NO 1 and a fragment of the hCG β-subunit β5 as hCGβ-subunits, then the medicament contains additionally so many αCG subunits according to SEQ ID NO 9 or SEQ ID NO 10 or glycan-linked oligopeptide fragments of αCG subunits that each βhCG subunit or each fragment of a βhCG subunit can form a heterodimer with an αCG subunit or a fragment of an α-subunit.

The subunits used according to the present invention of the human choriongonadotropine comprise in this connection for example choriongonadotropine isolated from natural sources, recombinant-produced forms as well as deglycosylated, non-glycosylated, modified glycosylated and other forms. The βhCG subunit β6 according to SEQ ID NO 1, SEQ ID NO 2 or SEQ ID NO 3 or SEQ ID NO 4 or the βhCG subunit β7 according to SEQ ID NO 5 or SEQ ID NO 6 as well as the βhCG subunits β5, β3 and β8 according to SEQ ID NO 7 or SEQ ID NO 8 of the human choriongonadotropine or the glycan-linked oligopeptide fragments contained in the medicament according to the invention are preferably produced by recombinant methods. When additionally the αCG subunit of the human choriongonadotropine according to SEQ ID NO 9 or SEQ ID NO 10 or fragments thereof are contained in the medicament according to the invention, they are preferably also produced by recombinant methods.

The gene-technological production of human gonadotropines is described as a standard procedure for recombinant FSH and trophoblastic hCG. In this connection, suitable cells (for example, ovary cells of the Chinese hamster—CHO cells) are transfected with cloned βhCG and αhCG DNA sequences and the protein that is produced by these cells is isolated. Up to now, eukaryotic cell lines, for example, ovary cells of the Chinese hamster—CHO cells, insect cell lines, are preferred for the expression of the protein for the gene-technological manufacture.

Preferably, mammal epithelium cell lines, preferred human epithelium cell lines, in particular preferred of the endometrium or the decidua, are used for expression.

Because of its complex structure, the integrity of the hCG molecule should be ensured in the isolation of the αCG and βhCG DNA fragments. Serine-O-bonded and asparagine-N-bonded glycosaccharide side chains (glycans) and optionally also disulfide bridged forms guarantee the biological activity of hCG.

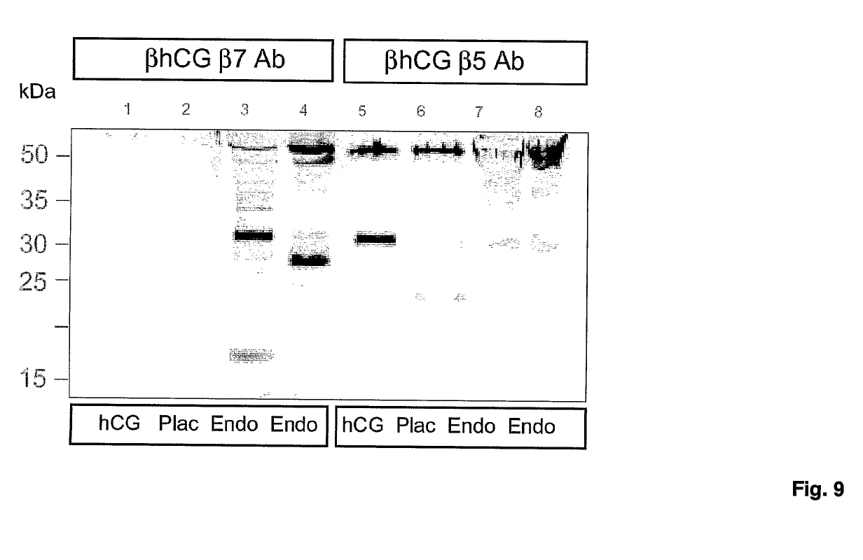

In up to now unpublished western blot tests regarding endometrial hCG (FIG. 9) we have been able to detect several glycosylated and partially deglycosylated βhCG molecule forms in analogy to the trophoblastic or placental hCG of gestation. Comparable to the placental hCG pattern of 56, 44, 38, and 35 kDa for the glycosylated and partially glycosylated αβ-dimeric hCG and of 32, 29, 24, 21 and 17 kDa for the glycosylated and partially glycosylated βhCG we were able to detect in western blot for the first time also the identical molecular hCG forms of endometrial origin. Different molecular forms of the glycosylated αCG of 24 and 21 kDa have been found also for the endometrium.

The alpha-subunit (α-hCG, αCG) is preferably N-glycosylated on the amino acids Asn-52 and/or Asn-78 of the ripe, mature amino acid sequence (SEQ ID NO 10) or Asn-76 and/or Asn-102 of the precursor (SEQ ID NO 9) and forms N-glycan chains with specific sugar residue portions.

The mature (ripe) endometrial or decidual β-subunits hCG β6 (SEQ ID NO 3, SEQ ID NO 4) and hCG β7 (SEQ ID NO 6) are preferably N-glycosylated on the amino acids Asn-13 and/or Asn-30 and preferably O-glycosylated on at least one of the CTP positions Ser-121, Ser-127, Ser-132, and Ser-138.

The precursor hCG 1-subunit 16 according to SEQ ID NO 1 or SEQ ID NO 2 or β7 according to SEQ ID NO 5 is preferably N-glycosylated on at least one of the following amino acids Asn-33, Asn-50 and/or O-glycosylated at Ser-141, Ser-147, Ser-152, Ser-158.

The (up to) two Asn-N glycan chains of the αhCG-subunit and β6-hCG-subunit or β7-hCG subunit are preferably provided with three or two antennae and tri, di, mono or non-sialysed. The Asn-N glycan chains each contain preferably 2 to 15, especially preferred 4 to 10 sugar residues, preferred with decreasing proportion of NAc glucosamine, sialic acid, galactose, mannose.

The (up to four) Ser-O glycan chains of the CTP region in the β6 or β7-hCG-subunit contain preferably 2 to 10 sugar residues, especially preferred 4 to 8 sugar residues, with four to two antennae and more strongly sialysed, preferably with decreasing proportion of sialic acid, NAc galactosamine, galactose, mannose, fucose.

The ripe mature alpha subunit αhCG contains particularly preferred 3 disulfide bridge bonds between the cysteine pairs AS 10-60, AS 28-82, and AS 59-87 as well as additional 2 preferred SH bridges between the AS 7-31 and AS 32-84.

The ripe mature beta-subunit β6-hCG or β7-hCG contains particularly preferred 2 disulfide bridge bonds between the cysteine pairs AS 9-57 and AS 38-90 as well as additional 4 preferred SH bridges between AS 23-72, AS 26-110, AS 34-88 and AS 93-100.

The preferred disulfide bridge bonds in the dimer hCG are responsible for formation of the typical cysteine knot structure that can be found analogously in a series of cysteine knot proteins. On the other hand, changed conditions of the SH bridge bonds in the hCG exhibit only minimal changes of biological activity.

In an up to now unpublished primary cell culture test it was found on the transcription as well as translation level that in endometrial cell culture the epithelial formation of βhCG subunits and αCG subunits is induced by means of mediators such as estradiol, progesterone, hCG, LPS, and Th2 cytokines and reduced by inhibitors such as Th1 cytokines, cycloheximide, and actinomycin D. This means for producing dimer epithelial hCG with the beta-subunit hCG β6 or hCG β7 that for this process preferredly epithelium cells of the secretorily transformed endometrium are used or epithelial endometrium cell lines are used that are capable of secretory transformation. Epithelial endometrium cell lines of cancerous origin are at least to be excluded or must be checked in regard to not expressing additional beta-subunit hCG β5, β8 and β3.

The medicament according to the invention is for example administered by injection. An especially preferred embodiment of the medicament is matched such that the parenteral administration of the medicament is enabled. For this purpose, preferably the precursor hCG or mature hCG with the subunit hCG β7 according to SEQ ID NO 5 or SEQ ID NO 6 or the precursor or mature hCG β6 according to SEQ ID NO 1 to SEQ ID NO 4 or glycan-linked oligopeptide fragments thereof are dissolved in an injection solution and transferred to provide a prefilled syringe.

Preferably, the precursor of the αCG subunit of the human choriongonadotropine according to SEQ ID NO 9 or the mature αCG subunit of the human choriongonadotropine according to SEQ ID NO 10 or glycan-linked oligopeptide fragments thereof are administered additionally.

The medicament is, for example, administered subcutaneously, intramuscularly, intramnially, sublingually, intrathecally or intravenously. Under emergency conditions the intravenous administration is preferred. In the case of a disorder of the early pregnancy such as implantation disorders, early pregnancy losses, imminent or habitual abortion, the administration of the medicament is preferably done by subcutaneous injection.

The endometrial hCG dosage to be administered depends on the state of the disease and the specific patient to be treated. Preferably, the medicament is adjusted such that the quantity of the human choriongonadotropine to be administered is 1 to 10 μg, especially preferred 3 to 6 μg, per kg body weight per day. Preferred individual doses are 50 μg to 1,000 μg of the disclosed hCG.

For parenteral administration of the medicament according to the invention, for example, 250 micrograms of the mature hCG formed of an endometrial β-subunit (β7 according to SEQ ID NO 6 with the mature hCG β6 according to SEQ ID NO 2 or SEQ ID NO 4) and an α subunits (SEQ ID NO 9 or SEQ ID NO 10) are dissolved in 0.5 ml of an injection solution and transferred to provide a prefilled syringe.

The invention concerns further a method for treatment of fertility and pregnancy disorders or for induction of an immunological tolerance in patients with autoimmune diseases or transplant processes, wherein a precursor hCG βsubunit of the human choriongonadotropine is selected from hCG β6 according to SEQ ID NO 1 or SEQ ID NO 2, hCG β7 according to SEQ ID NO 5 or a mature hCG βsubunit selected from hCG β6 according to SEQ ID NO 3 or SEQ ID NO 4, hCG β7 according to SEQ ID NO 6 or glycan-linked oligopeptide fragments of these sequences is administered to a patient.

Preferably, additionally the precursor of the αCG subunit of the human choriongonadotropine according to SEQ ID NO 9 or the mature αCG subunit of the human choriongonadotropine according to SEQ ID NO 10 or glycan-linked oligopeptide fragments thereof are administered.

Preferably, the quantity of human choriongonadotropine to be administered is 1 to 10 μg, particularly preferred 3 to 6 μg, per kg body weight per day, respectively. Preferred individual doses are 50 μg to 1000 μg hCG.

The injections with the medicament according to the invention are administered for imminent premature birth, in case of preeclampsia or intrauterine growth retardation e.g. daily, in the case of imminent premature birth with the beginning of regular labor. After labor has abated, the injection with the medicament according to the invention is carried out in intervals of 2 to 4 days.

In the case of acute onset of labor with advanced dilation of the cervix, the administration of the medicament according to the invention by intravenous infusion is preferred. In this connection, the protein dimer—hCG β7/α and/or hCGβ6/α (mature hCG β7 according to SEQ ID NO 6 or mature hCG β6 according to SEQ ID NO 2 or SEQ ID NO 4 with hCG α SEQ ID NO 9 or 10)—is dissolved in an infusion solution and administered over a time period of preferably four hours. Preferred dosage: 500 μg to 1,500 μg, preferably 1,000 μg, hCG β7/α or hCGβ6/α in 500 ml infusion solution.

Alternatively, the injection of hCG β7/α or hCG β6/α is done intraamnially. Preferred dosage: 500 μg to 1,500 μg, preferably 1,000 μg, hCG β7/α or hCGβ6/α.

For treatment of autoimmune diseases and for induction of immunological tolerance in case of transplant patients, preferably mononuclear cells are removed from the patient, incubated with the above mentioned hCG forms in vitro and subsequently administered subcutaneously, intravenously or locally to the patient, respectively. In this step, the mononuclear cells (primary monocytes, NK-cells or T-cells) are changed with regard to their properties such that they effect immune tolerance.

In this connection, the incubation of mononuclear cells with hCG in vitro induces the generation and secretion of hCG in these cells. This effect can be mainly detected in monocytes and NK-cells. A systemic hCG administration also acts by means of this effect.

Maintaining this immunity can be achieved by intravenous and local application of the aforementioned hCG forms or their fragments. By local hCG application (location of transplantation, joint gap, bladder, intestine, skin, liquor) in the form of injection, instillation, cremes, sprays or capsules, the chemotactic effect of the hCG on the mononuclear cells that induce immune tolerance is taken advantage of.

Preparation: According to the prior art the preparation of a recombinant trophoblastic hCG is done in a culture of ovary cell lines of the Chinese hamster (CHO) cells with CHO-DUKX fibroblast cells, COS-7 cells or further CHO cell lines described in the literature (Chappel et al., 1992; Matzuk et al., 1989; Garcia-Camayo et al., 2002; Birken et al., 2003). In this connection, the αgene was transfected like the βgenes of the trophoblast.

The inventors have found that the glycolization of the recombinant hCG produced according to the prior art differs disadvantageously from the hCG naturally expressed in the human endometrium and decidua. The glycolization of hCG has been found to be surprisingly epithelium-specific. In this connection, the glycolization has strong effects on the specificity and the biological activity of the hCG.

Also, the inventors were able to list for the first time by sequence analysis the exact βhCG nucleotide sequence of exon 1 and exon 2 that in the healthy secretorily transformed endometrium and decidua of the human are expressed as RNA hCG β7 or hCG β6 or hCG β7+β6 (see SEQ ID NO 11 to 13).

The invention concerns therefore further a method for producing human hCG with the subunits α-choriongonadotropine (αCG) and β-human choriongonadotropine (βhCG) in isolated human epithelium cells or epithelium cell lines of endometrial or decidual origin. Preferably, the expressed β-subunit in this connection is comprised of βhCG β6 and/or βhCG β7.

In comparison to prior procedures, the preparation according to the invention with the goal of using natural or artificial human epithelium cells has the advantage of producing an epithelium-specific hCG secretion product with epithelium-specific glycolization that is more pronounced in the human epithelium. It avoids moreover the disadvantages that were caused according to the prior art in that the hCG in the past was produced in a mammalian cell culture without immediate relation to natural human epithelium-specific glycolization program.

The isolated endometrial and decidual epithelium cells are preferred cell lines of human origin. Derived endometrial epithelium cell lines or epithelium cell lines with additional transfection of βhCG genes β6 and β7 and/or further βhCG genes (β5, β3, β8, β1, β2) and/or genes for glycolization of the hormone are expressly included here. The epithelium cells are preferably harvested from native endometrial tissue.

Advantageously, the hCG expressed in these cells has the above-mentioned preferred glycolization pattern and the above-mentioned disulfide bridges.

With the inventive method it is therefore possible to make available hCG whose glycolization, folding and disulfide bridges correspond to natural features.

Further applications of the medicament according to the invention will be disclosed in the following.

For the treatment of sepsis hCG (comprised of α-CG and β6/7-hCG or the glycolized oligopeptides) is infused, preferably daily. Preferred dosage: 500 to 1,000 μg/d hCG. Virus-caused carcinoma and sarcoma are systemically and locally treated with a dosage of preferably 1,000 μg/d.

For use in contraception (prevention of pregnancy) hCG (comprised of α-CG and β6/7-hCG or the glycolized oligopeptides) is injected subcutaneously or administered sublingually. Preferred dosage: 10 μg hCG daily. Alternatively, the hCG is administered subcutaneously by means of rods of polymer or intravaginally by means of rings of polymers. The rods or rings are comprised preferably of polyethylene-co-vinyl acetate and release preferably 2.5 to 20, preferably 4 to 7 μg hCG (comprised of α-CG and β6/7-hCG) daily.

As prophylaxis of an HIV infection hCG-containing gels and cremes that contain hCG (comprised of α-CG and β6/7-hCG or the glycolized oligopeptides) in concentrations of preferably 1% are used.

For treatment of severe tissue ischemia such as apoplexia, heart attack, or severe postpartum brain edema of newborns hCG (comprised of α-CG and β6/7-hCG or the glycolized oligopeptides) is preferably infused or, in case of burns, applied locally in the form of sprays. Preferred dosage: 500 to 1,000 μg daily.

For treatment and prophylaxis of an allergic-inflammatory reactions of the upper air passages (hey fever, bronchial asthma), hCG (comprised of α-CG and β6/7-hCG or the glycolized oligopeptides) is preferably administered in the form of a spray (alternatively, a creme or gel). Preferred dosage: 50 to 100 μg daily.

For treatment of benign prostate hyperplasia (BPH) hCG (comprised of α-CG and β6/7-hCG or the glycolized oligopeptides) is prescribed preferably sublingually. Preferred dosage: 0.5 μg twice a day.

Treatment of autoimmune disease of the eye: in case of autoimmune uveitis preferably in intervals of 4 to 7 days a solution comprised of containing α-CGE and β67-hCG or the glycolized oligopeptides is injected. Preferred dosage: 0.5 ml with 10 μg/ml hCG. This therapy can also be prescribed in case of a therapy-resistant glaucoma therapy or in the case of danger of rejection of a cornea transplant.

In patients with multiple sclerosis hCG (comprised of α-CGE and β6/7-hCG or the glycolized oligopeptides) is instilled preferably intrathecally on a weekly basis. Preferred dosage: 2 ml in a concentration of 10 μg/ml.

Treatment of Crohn's disease and colitis ulcerosa: oral administration of hCG comprised of α-CG and β6/7-hCG or the glycolized oligopeptides in a biomembrane capsule that releases hCG only once it reaches the intestine or colon, preferably in a concentration of 5 μg/ml.

For transplantation of autologous and/or xenogenic islet cells they are preferably stimulated before transplantation preferably for 24 to 72 hours, 48 hours, in an hCG emulsion with 2 μg/ml hCG and subsequently intravenously injected. Alternatively, autologous or xenogenic islet cells are encapsulated in an hCG-releasing biomembrane, made preferably of a biodegradable polymer such as poly(ε-caprolactone) (PCL) and implanted in this way.

For the treatment of treatment of interstitial cystitis and chronic cystitis preferably a biodegradable implant, preferably of poly(ε-caprolactone) (PCL), in the form of a rod that continuously releases hCG is inserted in case of chronic cystitis or interstitial cystitis.

Treatment of HIV infection: In case of pronounced T-cell drop in the context of HIV infection hCG (comprised of α-CGE and β6/7-hCG or the glycolized oligopeptides) is injected preferably intravenously. The administration is carried out preferably daily for 2 weeks. Preferred dosage: 1,000 μg/ml.

With the aid of the following Figures and embodiments the invention will be explained in more detail. In this connection, the Figures show substantial analogies and specific differences in the molecular organization structure of the placental or endometrial hCG formation. Tests with regard to gene expression, sequence analysis, hormone assays in tissue, molecular detection of specific hCG antibodies (western blot) and immune histochemical methods affirm the cycle-dependent hCG formation in the healthy secretory endometrium.

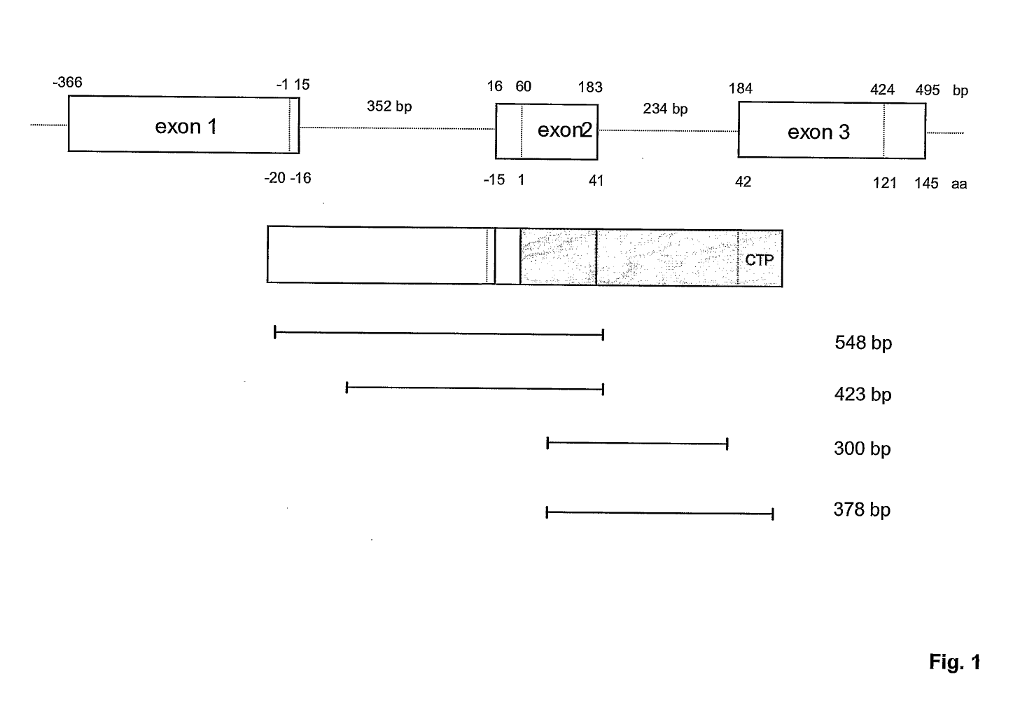

FIG. 1: organization of βhCG genes β5, β8, and β3 as well as of βhCG genes β6 and β7;

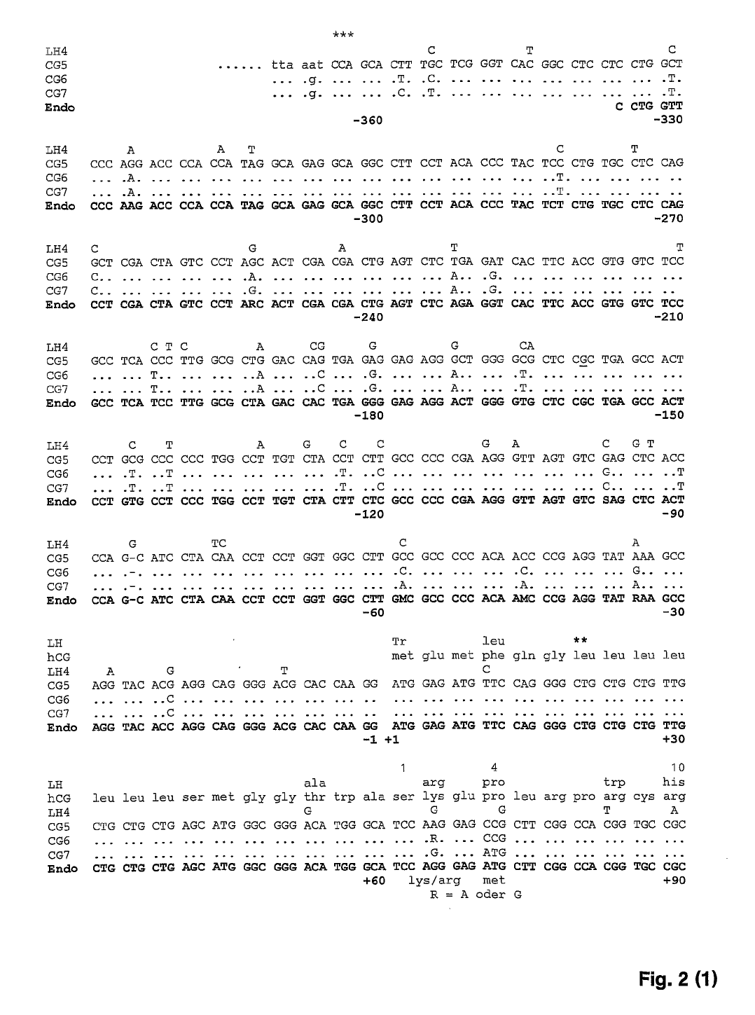

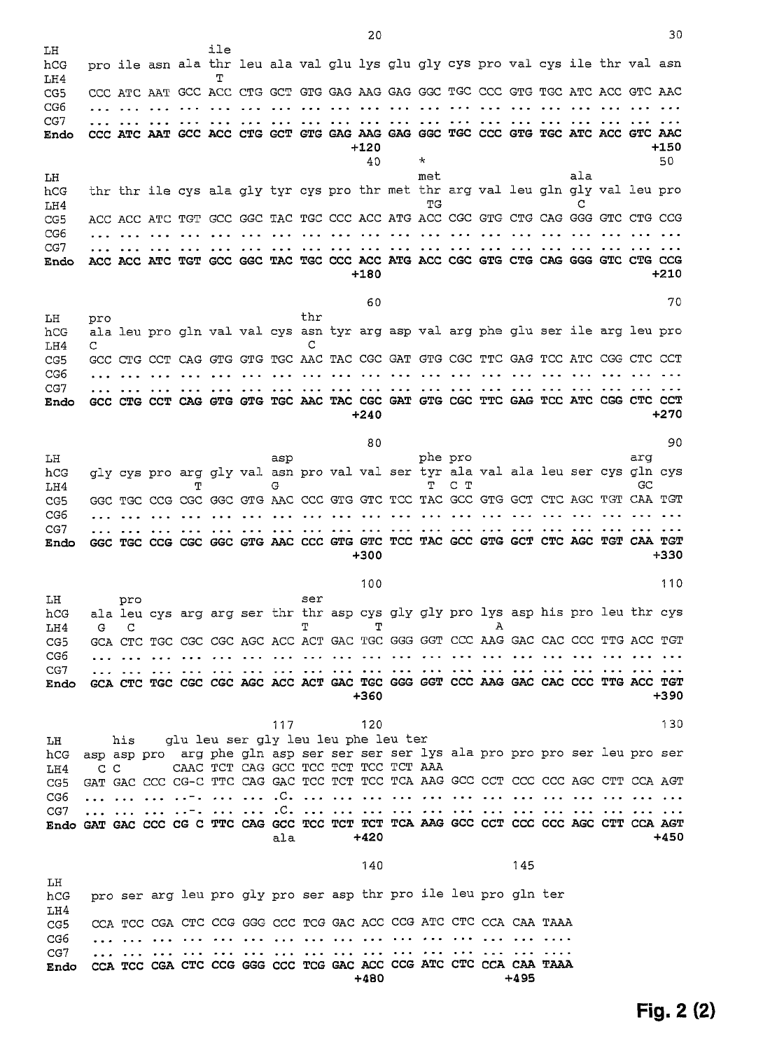

FIG. 2 gene expression and nucleotide sequence of mRNA of βhCG genes β5, β6, and β7 and of βLH gene β4 in connection with coded amino acid sequence;

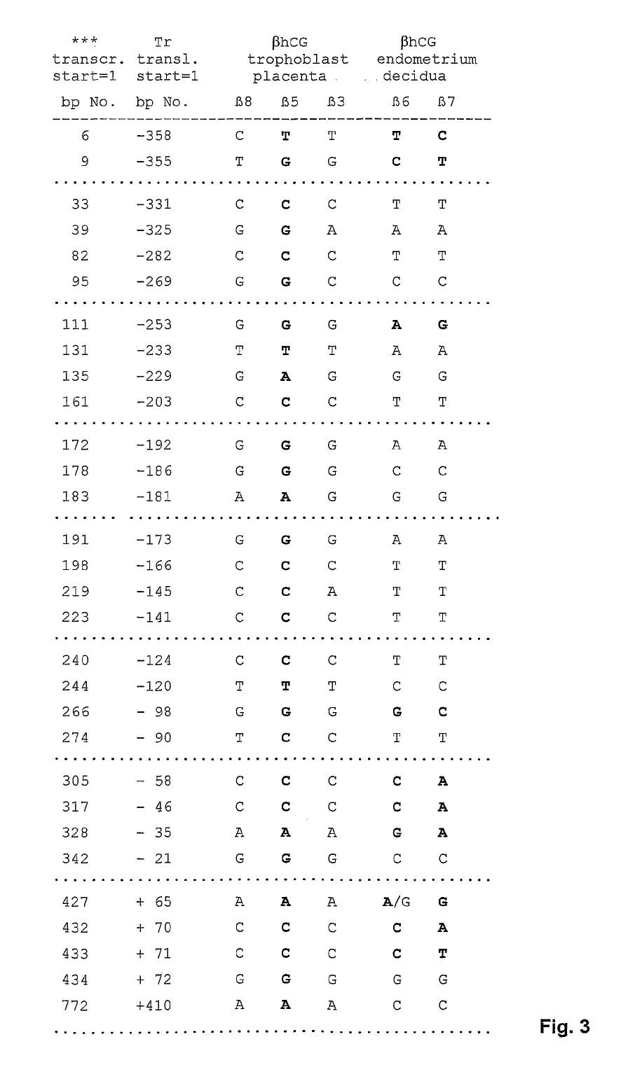

FIG. 3 localization of different nucleotide sequences of βhCG mRNA of gene β5, β6, and β7 in exons 1, 2 and 3;

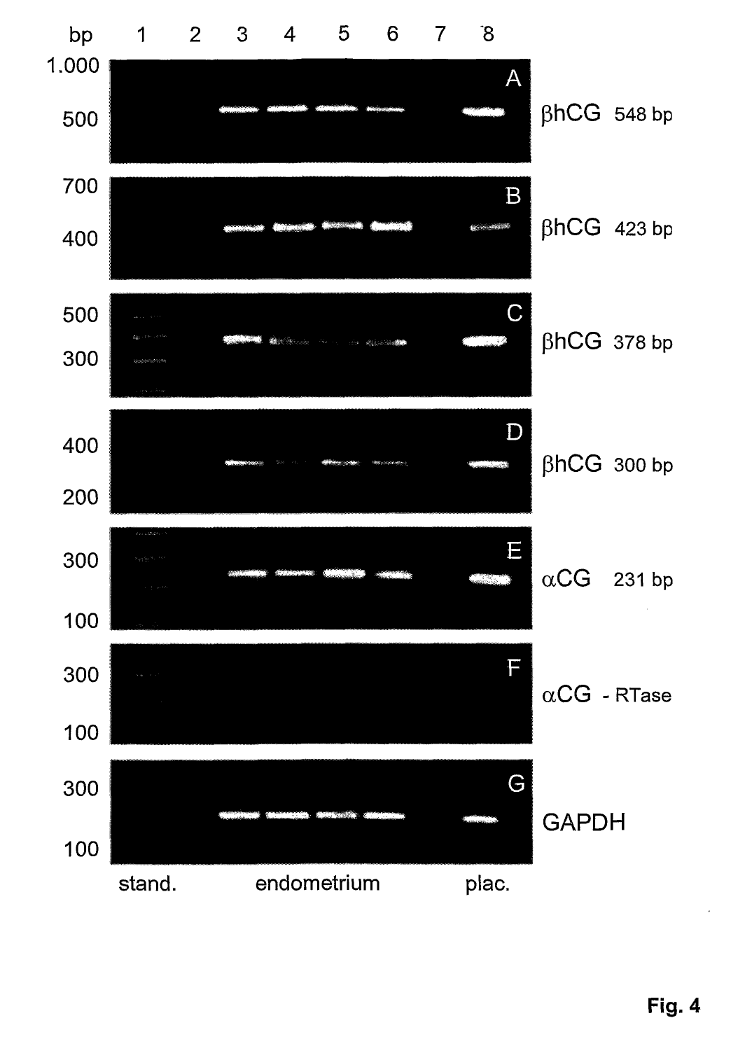

FIG. 4 gene expression of βhCG and αCG after RT-PCR in secretory endometrium;

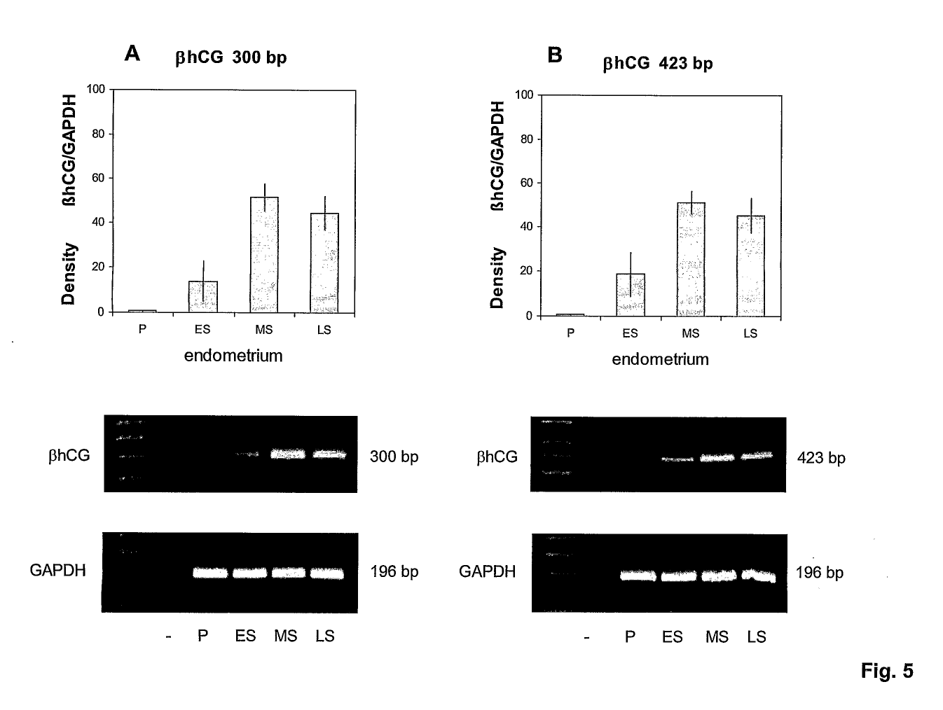

FIG. 5 cycle-dependency of the endometrial gene expression of βhCG;

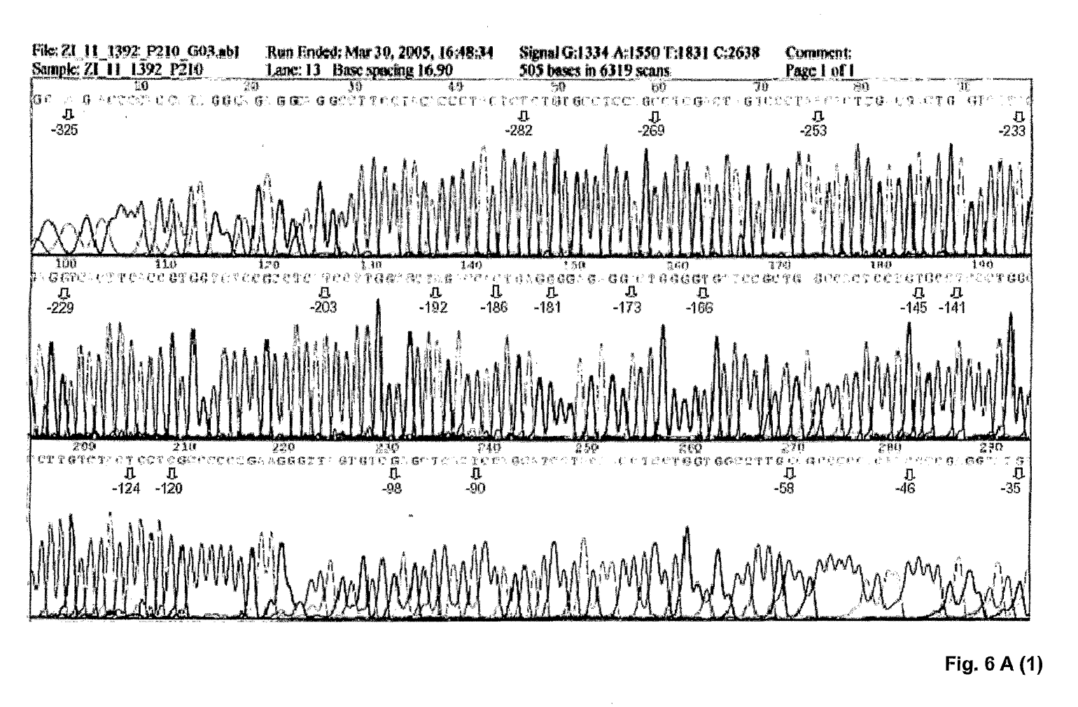

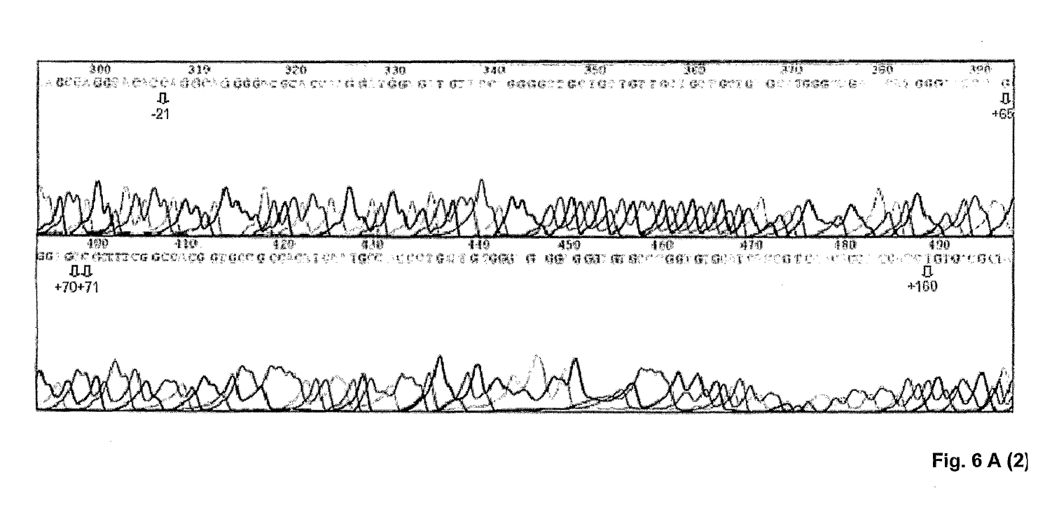

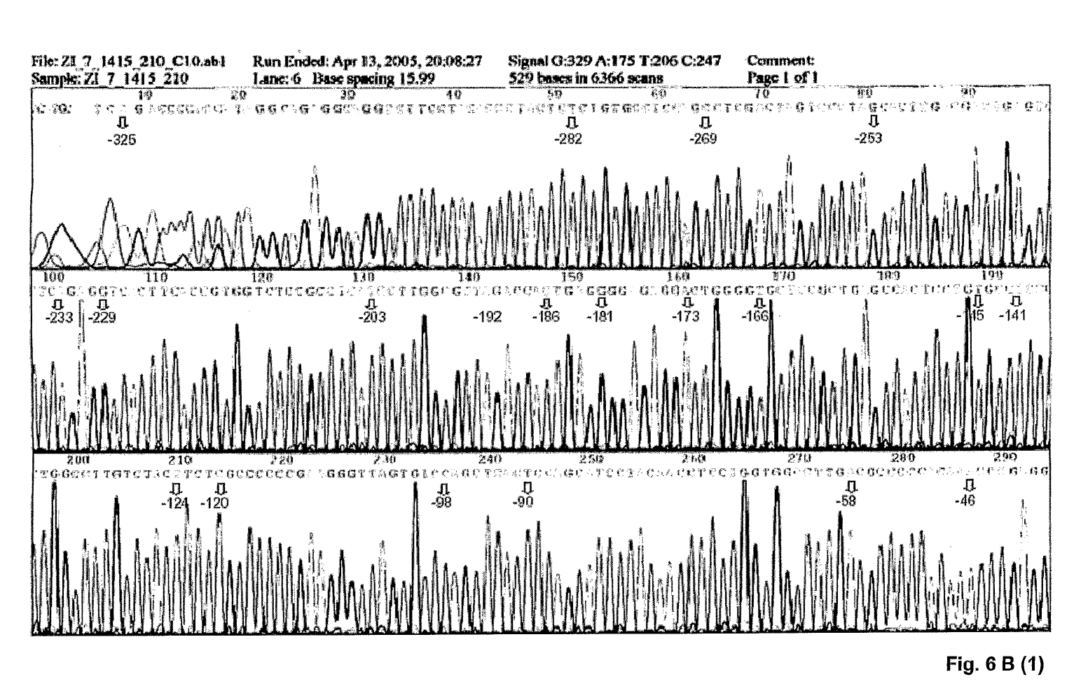

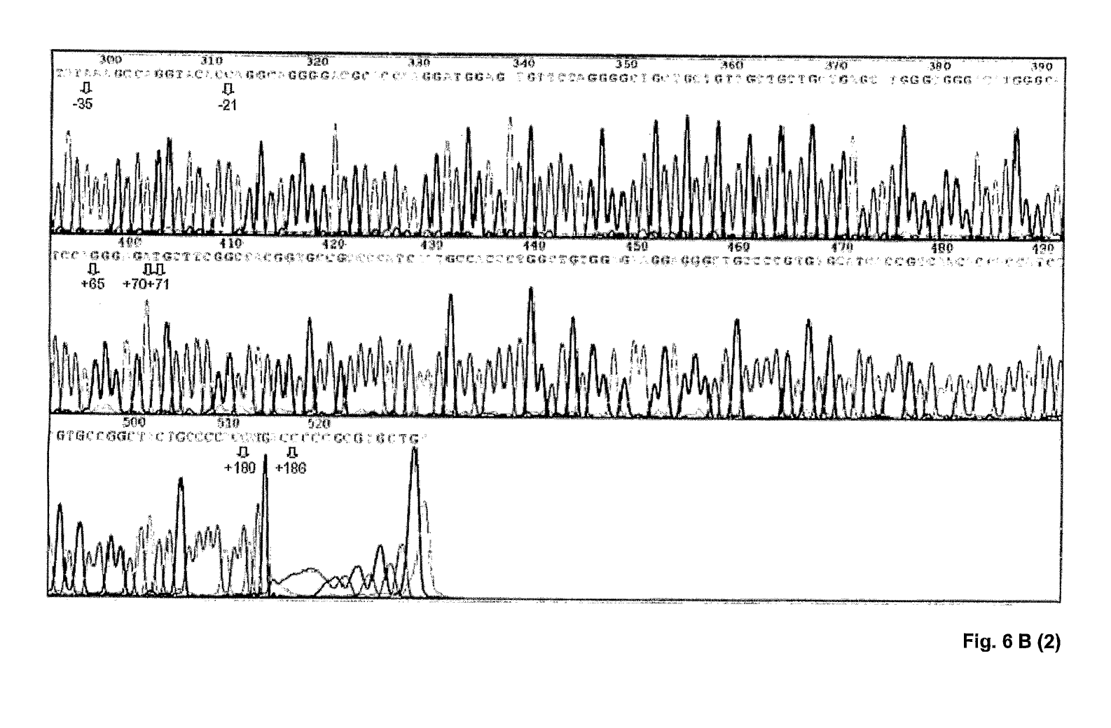

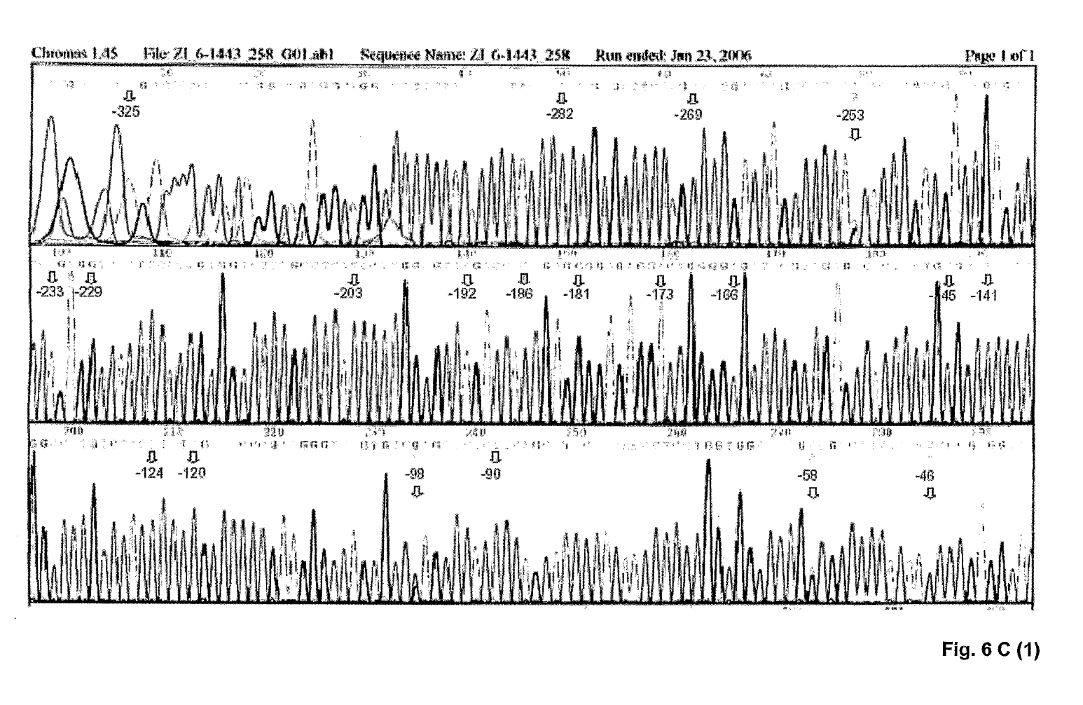

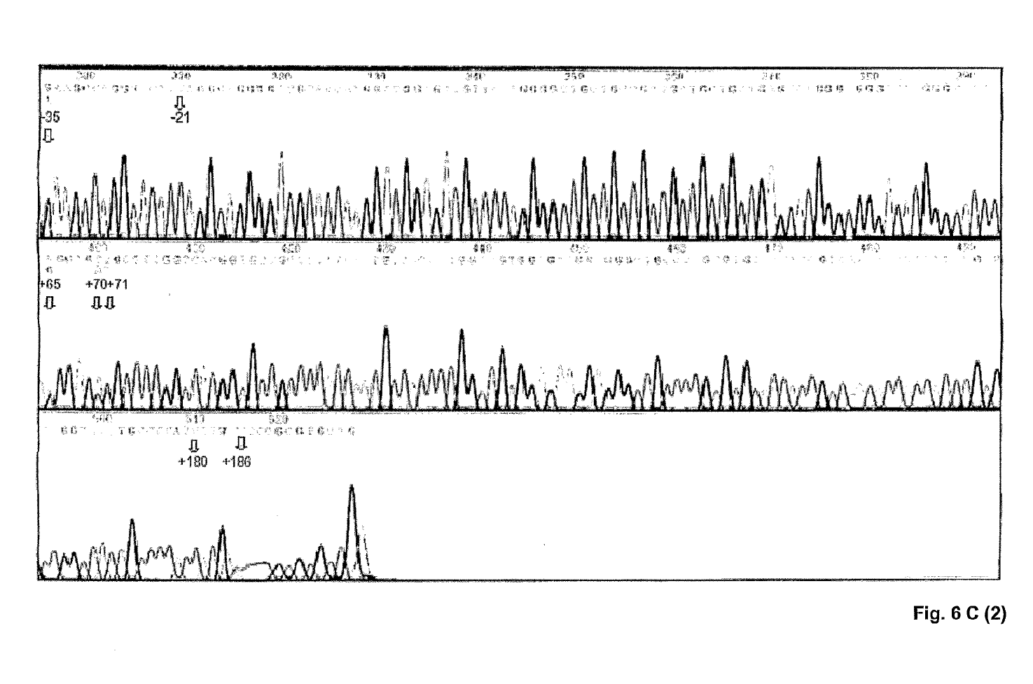

FIG. 6(A) sequence analysis of the transcript of the endometrial gene expression βhCG β6,
(B) sequence analysis of the transcript of the endometrial gene expression βhCG β7,
(C) sequence analysis of the transcript of endometrial gene expression βhCG β6 and βhCG β7;

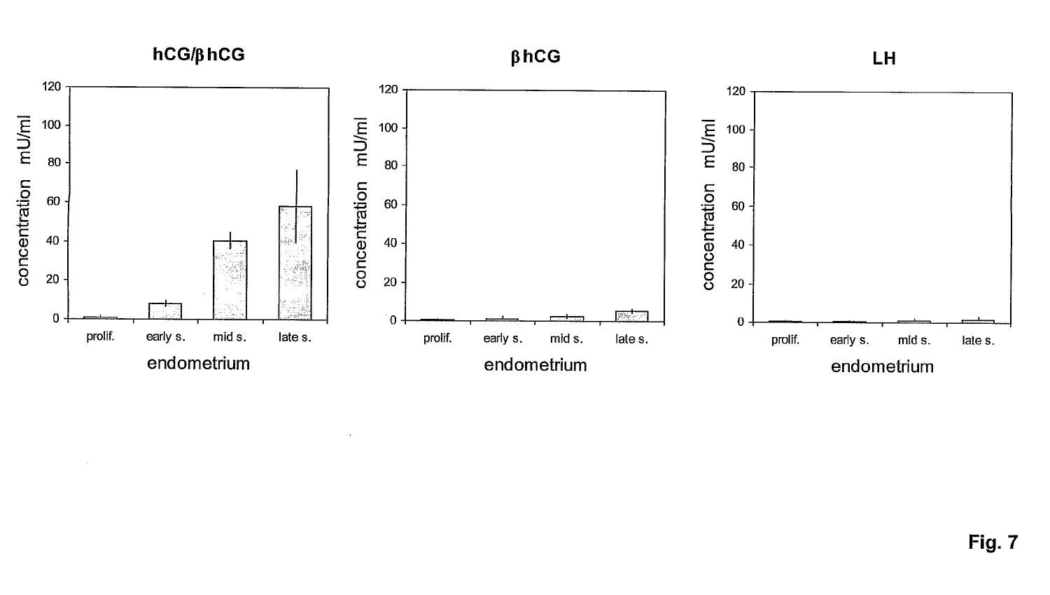

FIG. 7 concentration determination of the endometrial hCG in the endometrium homogenate;

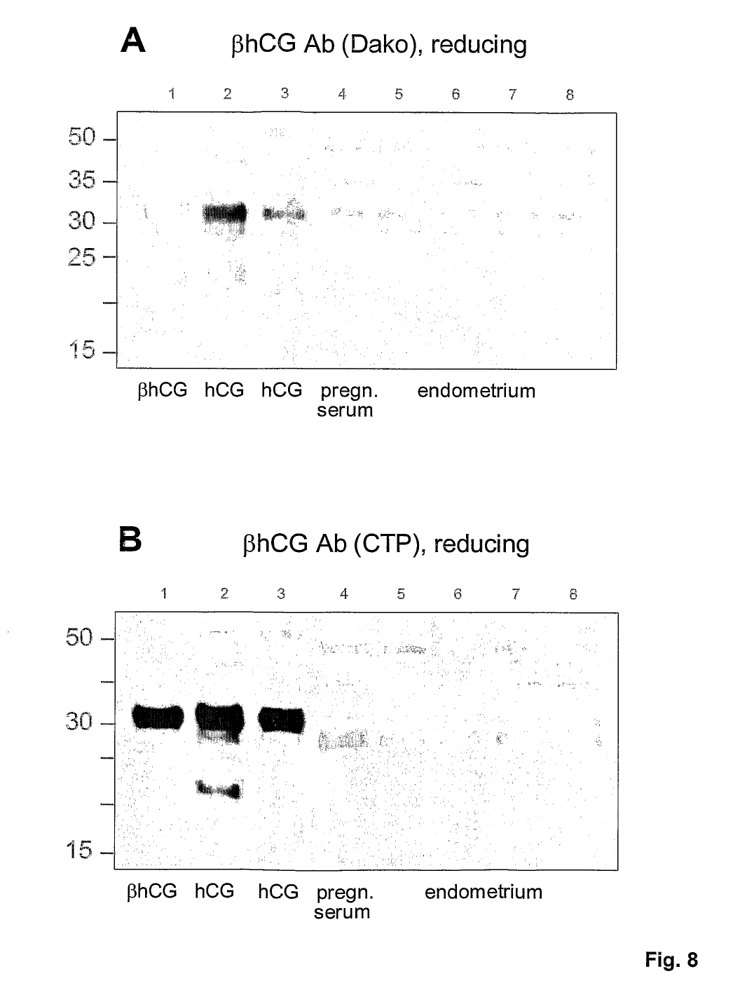

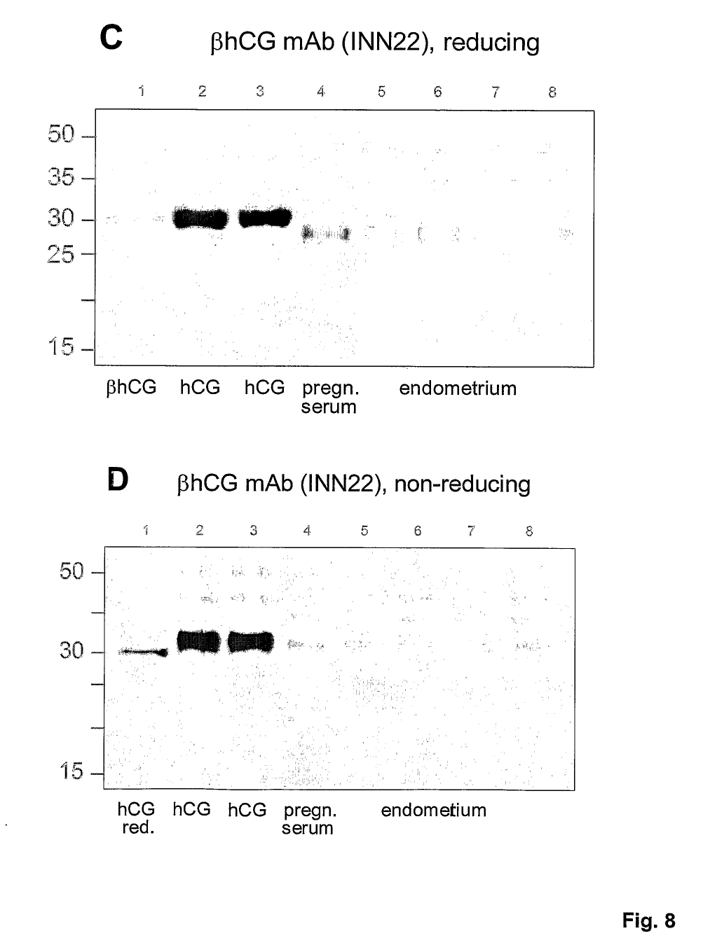

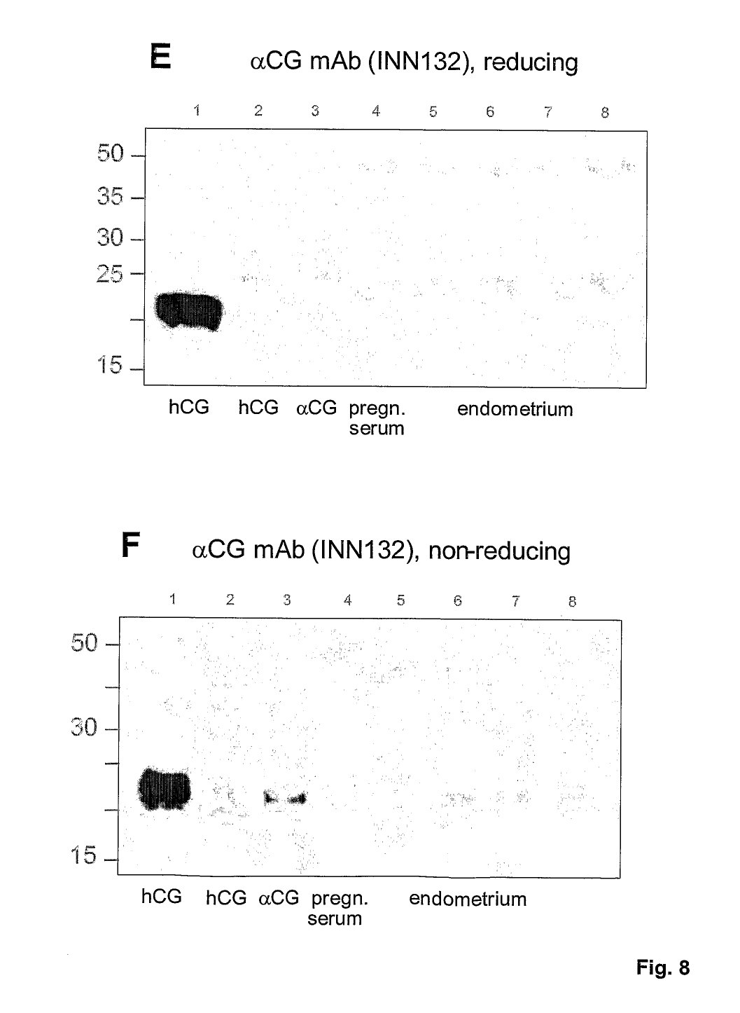

FIG. 8 western blot test in regard to molecular structure and glycolization of the placental and endometrial hCG with
(A, B) polyclonal hCG and CTP-hCG antibody,
(C, D) monoclonal βhCG antibody,
(E, F) monoclonal αCG antibody;

FIG. 9 western blot test for differentiation between hCG of endometrial (β7, β6) and trophoblastic (β5) origin.

The organization of βhCG placental genes β5, β38 and β33 as well as of the endometrial genes β6 and β7 is illustrated in FIG. 1. The genes of the βhCG subunit are comprised each of three exons and two intervening introns. Exon 1 comprises the promoter sequence, the two structure genes exon 2 and exon 3 including the C-terminal peptide (CTP) code the ripe (mature)βhCG subunit with 145 amino acids (aa). The βhCG gene is expressed from bp −366 in exon 1 (transcript start ***) through bp+1 in exon 1 (translation start, Tr) up to bp +495 in the CTP of exon 3. Exon 1 covers the bp region of −366 to +15, exon 2 from +16 to +183, and exon 3 from +184 to +495. Four intron-bridging βhCG primer pairs with the resulting amplicons of 548, 423, 378 and 300 bp affirm the full-length βhCG gene expression.

The βhCG gene β1 and β2 contain a point mutation in the donor splice site of the first intron. An mRNA resulting from alternative splicing of intron 1 codes proteins of 132 amino acids whose sequences however have no similarity to the amino acid sequences of the other βhCGs (Policastro et al., 1983; Talmadge et al., 1984; Bo and Boime, 1992).

In FIG. 2 the different nucleotide sequences of the placental βhCG gene β5 (CG5) are compared to the epithelial βhCG genes β6 and β7 (CG6, CG7) and the βLH gene β4 (LH4) as well as the detected endometrium sequences (endo). Also, the correlated different amino acid sequences in the protein molecule are listed. The mRNA of βhCG genes comprises the nucleotide region of −366 to +495 bp, the nucleotides from +1 to +496 bp code the prehormone (βhCG precursor) with 165 amino acids, the nucleotides from +60 to +495 bp code the ripe (mature) β-subunit with 145 amino acids. In the Table, in the endometrium sequence M represents C or A, R represents G or A, and S represents G or C. The start of exon 1 is identified at ***, that of exon 2 at **, and that of exon 3 at *.

FIG. 3 shows the differences in the nucleotide sequences of exon 1 (bp −358 to −21, n=25), exon 2 (bp+65 to +71, n=3), and exon 3 (bp+410, n=1). Promoter gene and the structure genes as a whole differ between genes hCG β5 and hCG β7 in 27 nucleotide positions, the hCG gene β7 differs from the hCG β6 in 10 nucleotide positions. The nucleotide sequences of the hCG subunits β3, β5, and β8 in the promoter gene show several differences, the amino acid sequences of the prehormone and mature hCG subunits β3, β5 and β8 are however identical. Numbering of the bp numbers in FIG. 3 is related to the transcription start or translation start.

As can be seen in Table 1 and the preceding Figure, the resulting amino acid sequence of the placental hCG does not differ despite its plural βhCG β5, β8. β3 subunit structures. For the endometrial hCG with its βhCG β7 and β6 subunits, in addition to the amino acid +117 in the C-terminal region, further amino acids in the N-terminal region are however changed relative to the placental hCG.

These subunits αCG and βhCG, expressed by different genes, combine intracellularly soon after protein synthesis in the endoplasmic reticulum and experience post-translatory modifications into the specific, biologically active heterodimer form (disulfide bridge bonds and glycolization in the endoplasmic reticulum, heterodimerization, seatbelt configuration, and prehormone cleavage in the Golgi apparatus). The resulting amino acid sequences of the mature hCG β7 subunit and of hCG β3, β5, β8 subunits differ in the amino acid positions +2 (arginine/lysine), +4 (methionine/proline), and +117 (alanine/aspartate), those of hCG β6 as well as hCG β7 with the hCG 5 subunit also in the amino acid position +117 (alanine/aspartate) and that of hCG β6 and βhCG β7 subunit also in the amino acid positions +2 (lysine/arginine) and +4 (proline/methionine). Moreover, in position +2 for hCG β6 arginine can be represented with the nucleotide sequence AGG (b). The differences in the amino acid sequence are combined in Table 1.

TABLE 1

| AS position | hCG β5 | hCG β7 | hCG β6 |
|---|---|---|---|
| +2 | Lys | Arg | Lys or Arg |
| +4 | Pro | Met | Pro |
| +117 | Asp | Ala | Ala |

In FIG. 4, we have been able for the first time to demonstrate with our lab results that in normal secretory endometrium of healthy women epithelial hCG is expressed. The gene expression of secretory endometrium comprises the α CG subunit as well as the full-length RNA of the βhCG subunit of exon 1 to exon 3 including the CTP region.

From tissue samples of the endometrium and the placenta RNA was isolated as a control by trizol extraction and analyzed by means of semi-quantitative RT-PCR with primer pairs that specifically recognize βhCG (A-Din FIG. 4), α hCG (E, F in FIG. 4), and GAPDH (G in FIG. 4). For this purpose, the primer pairs listed in Table 2 were used in RT-PCR under standard conditions:

TABLE 2

| | | primer | | | | bp | No. |
|---|---|---|---|---|---|---|---|
| No. | gene | location | exon | strand | primer sequence | amplicon | paired |
| 1 | βhCG | −353/−337 | 1 | sense | 5′-TCGGGTCACGGCCTCCT-3′ | 548 | 4 |
| 2 | βhCG | −229/−209 | 1 | sense | 5′-TCACTTCACCGTGGTCTCCG-3′ | 423 | 4 |
| 3 | βhCG | 108/127 | 2 | sense | 5′-GGCTGTGGAGAAGGAGGGCT-3′ | | 5, 6 |
| 4 | βhCG | 197/178 | 2, 3 | anti-s. | 5′-CAGCACGCGGGTCATGGT-3′ | | 1, 2 |
| 5 | βhCG | 406/384 | 3 | anti-s. | 5′-GAAGCGGGGGTCATCACAGGTC-3′ | 300 | 3 |
| 6 | βhCG | 484/468 | 3 | anti-s. | 5′-TCGGGGTGTCCGAGGGC-3′ | 378 | 3 |
| 7 | αhCG | 83/102 | | sense | 5′-TGCAGGATTGCCCAGAATGC-3′ | 231 | 8 |
| 8 | αhCG | 313/294 | | antis. | 5′-CCGTGTGGTTCTCCACTTTG-3′ | | 7 |
| 9 | GADPH | 335/352 | | sense | 5′-CCATGGAGAAGGCTGGGG-3′ | 196 | 10 |
| 10 | GADPH | 530/510 | | anti-s. | 5′-CCAAAGTTGTCATGGATGACC-3′ | | 9 |

The base pair length of the DNA products amplified by RT-PCR are listed as bp. The primers are contained as SEQ ID NO 15 to 24 in the attached sequence listing. The specific βhCG primers do not amplify βLH-mRNA.

In FIG. 4, the results of RT-PCR for four tissue samples of the endometrium are shown in the lanes 3 to 6 (“endometrium”) and in the lane 8 a tissue sample of “early gestation” placenta (“plac.”) is shown as a control. In lane 1 for size determination a DNA marker as a standard (“stand.”) is shown. In lane 2 a negative control (without primer, without RNA) is shown.

In FIG. 4A the βhCG-specific primers 1 and 4 of Table 2 have been used. The comparison with the DNA marker shows that the amplified DNA has the expected length of 548 bp. In FIG. 4B the βhCG-specific primers 2 and 4 of Table 2 have been used. A comparison with the DNA marker shows that the amplified DNA has the expected length of 423 bp. In FIG. 4C the βhCG-specific primers 3 and 6 of Table 2 have been used. The amplified DNA has the expected length of 370 bp. In FIG. 4D the βhCG-specific primers 3 and 5 of Table 2 have been used. The amplified DNA shows the expected length of 300 bp. In FIG. 4E and FIG. 4F the αCG specific primers 7 and 8 have been used. The DNA product has in FIG. E the expected length of 231 bp. Without revertase in the cDNA batch (-RTase) the reaction does not happen, i.e., is RNA and not endogenic DNA.

In FIG. 4E GAPDH-specific primers 9 and 10 of Table 2 have been used also as a control and for a semi-quantitative determination. The comparison to the DNA marker shows that the amplified DNA has the expected length of 196 bp. The results show that βhCG and αhCG mRNA in the secretory phase of healthy endometrium are expressed in approximately the same concentration as the placenta.

FIG. 5 shows that the βhCG mRNA expression depends on the differentiation level of the secretory transformation of the endometrium. The endometrium biopsies have been evaluated always after diagnostic curettage by experienced pathologists as a cycle-appropriate and as normal tissue of the proliferative phase up to the late secretory phase. The endometrial RNA was extracted and determined by RT-PCR semi-quantitatively relative to the corresponding GAPDH amplification. For the measurements patients of the proliferative (P, n=22), early secretory (ES, n=28), mid secretory (MS, n=26), and late secretory (LS, n=15) phase of the menstrual cycle were selected. A visual densitometric evaluation was performed (±SEM).

In FIG. 6 the results of the sequence analysis for confirmation of the gene expression of βhCG mRNA in the secretory endometrium is illustrated. The employed cDNA amplificats were used after RNA extraction of the endometrial tissue and after RT-PCR under standard conditions for sequencing. In FIG. 6A after sequencing of the βhCG amplificats a nucleotide sequence for βhCG β6, in FIG. 6B a nucleotide sequence for βhCG β7 and in FIG. 6C a nucleotide sequence for βhCG β7 with βhCG β6 are shown. The sequences confirm with high precision the nucleotide sequences compiled in Table 2 for the expression of the endometrial βhCG β7 subunit and βhCG β6 subunit. The detected nucleotide sequence is based on the knowledge of the cDNA amplificats employed for the tests with 548 bp through exon 1 and exon 3. With these results an mRNA sequence for the expression of an endometrial βhCG subunit is presented for the first time.

In addition to the endometrial transcription of both βhCG subunits we can also confirm the translation the translation of the endometrial hCG. The concentrations of endometrial hCG are detected cycle-dependent in the endometrium homogenates.

In FIG. 7 the hormone concentrations of total hCG/βhCG, free βhCG subunit and of LH in the endometrium homogenates are illustrated. The hormone concentrations were measured in the supernatant of approximately 100 mg tissue per ml buffer. The tissue samples were taken at different points in time of the menstrual cycle and used for the examinations (proliferate, n=19; early secretory, n=24; mid secretory, n=23; late secretory, n=10; ±SEM). The endometrial hCG increases with secretory transformation to values about 60 mU/ml while LH stays at basal values and the free βhCG subunits increase only minimally.

Under consideration of endometrial translation of an epithelial hCG we are presenting here in FIG. 8 for the first time results for SDS polyacrylamide electrophoresis and western blot tests in homogenates of the normal secretory endometrium. The four lanes each of the endometrium samples (lanes 4-8) are compared with commercially purified hCG preparations or αCG and βhCG subunits (lanes 1-3) and a pregnancy serum of the first pregnancy trimester.

In FIG. 8 AB the blots are treated with polyclonal hCG antibodies (Dako) and a polyclonal CTP hCG antibody (Biotrend) as primary antibodies. In FIG. 8 CD the blots are treated with a monoclonal βhCG antibody (INN22) under reducing or non-reducing conditions. In FIG. 8 EF the blots are treated with a monoclonal αCG antibody (INN132) under reducing or non-reducing conditions.

The endometrial tissue samples show predominant βhCG subunit bands of approximately 31 kDa or 29 kDa and αCG subunit bands of 24 kDa or 21 kDa as well as αβhCG dimer bands of 44 kDa, 38 kDa, and 35 kDa or further βhCG monomer bands of 24 kDa, 21 kDa, 17 kDa and 15 kDa depending on the desialysing or deglycolysing level.

In addition, up to now unpublished lab results of western blot in tissue homogenates of several secretorily transformed endometrium samples of women with healthy cycles are presented that confirm with polyclonal and monoclonal antibodies under reducing or non-reducing conditions differently glycolysed and partially deglycolysed molecular forms of the dimer αβhCG and of αCG subunit and βhCG subunit in comparison to placental hCG (FIG. 8).

Gene-specific βhCG antibodies that have been especially developed for the alternative detection of hCG β7 and β5 dimers confirm in western blot the precise detection based on endometrial or placental (trophoblastic) origin (FIG. 9).

By means of hCG-specific, βhCG-specific and αCG-specific antibodies, the cycle-dependent epithelial hCG secretion in the endometrial gland and luminal epithelium of a healthy women in tissue sections primarily of the mid secretory and late secretory phases can be unequivocally demonstrated with regard to immune histochemistry.

### EXAMPLE 1

### Gene Technological Preparation of Recombinant hCG

(similar to Loumaye et al., 1995; Howles, 1996; Matzuk et al., 1989; Carcia-Campoya et al., 2003; Birken et al., 2003)

### A. Preparation of Human DNA:

Isolation and characterization of the entire βhCG gene (promoter gene exon 1, structure genes exon 2 and exon 3 including the introns) for the βhCG genes 6 and β7 that are coding for the precursor and mature transcript, isolation and characterization of the entire αCG (hCG α)-gene that codes for the precursor and mature transcript.

B. Insertion of βhCG-DNA and αCG-DNA into a Vector (Plasmid Construction)

Use of TOPO TA cloning kit (Invitrogen or alternatively pGEM vector system Promega) according to manufacturers instructions each for hCG β6, hCG β7, and hCG α (SEQ ID NO 11 to SEQ ID NO 13 and SEQ ID NO 15) and insertion into the expression vector. The inserted DNAs are combined with the DNA sequence of dehydrofolic acid reductase (DHFR) that is required for synthesis of the ribonucleic acid precursors in DHFR-deficient mammalian host cells.

### C. Incorporation (Co Transfection) of βhCG and αCG Expression Vectors in the Mammalian Host Cell

The αCG and βhCG expression vectors are transfected by Ca coprecipitation into the well-characterized animal CHO (Chinese hamster ovarian) cell line that represent DHFR-deficient cells. For producing the epithelial and non-trophoblastic dimer form of the glycolysed hCG, the clones βhCG genes β6 or βhCG gene β7 are co-transfected and cultured together with αCG.

### D. Selection of Individual Clones According to the Following Criteria:

The clones each originating from one cell are checked with regard to their ability for forming hCG, their biological activity of hCG, and their genetic stability.

E. Establishing a Master Cell Bank (MCB) of an Individual Co-Transfected Cho Cell with αhCG and Respective βhCG Expression Vectors, which Cell Originates from a Clone That has been Evaluated as Optimal.

### F. Establishing a Working Cell Bank (WCB)

Established by proliferation of cells of a single MCB container.

G. Commercial Production of Recombinant hCG (r-hCG), Gene-Specifically:
proliferation of cells from the working bank (WCB)
culture expansion in glass vessels, rolling flasks
bioreactor: attachment and growth of the cells, hCG production, collection of hCG culture medium
   H. Fine Purification of the Recombinant hCG from the Culture Medium:
ultra filtration
chromatography by means of columns
immune affinity chromatography
chromatography
ultra filtration
purified raw products r-hCG (αhCG and βhCG-gene β6 or β7).

### I. Control of Batch-to-Batch Quality

combination of chromatography and MALD-TOF mass spectrometry
N-glycosidic and O-glycosidic glycoprotein side chains (glycan) characterization
glycan mapping method for batch control of the sialysing degree
complete dissolution of the N-glycan and O-glycan bonds at the αCG and βhCG molecules
obtaining batch-to-batch consistency for commercial production (analog to Gervais et al., 2003; Gam et al., 2003; Birken, 2005).

### EXAMPLE 2

Gene-technological production of recombinant αβhCG (βhCG gene β7-specific or gene β6-specific or gene β5-specific) in human epithelium cells of the secretory endometrium for the decidua:
Use of a TOPO TA cloning kit (Invitrogen or alternatively pGEM-T vector system Promega) according to manufacturer's instructions each for hCG α and hCG β6, β7 (SEQ ID NO 11 to SEQ ID NO 13 and SEQ ID NO 15) and insertion into the expression vector in accordance with Example 1.
Cell separation and culturing of human epithelium cells of the secretory endometrium for the decidua.
Incorporation (transfection) of the vectors in accordance with Example 1.
Utilization of native synthesis efficiency of the human epithelium cells of the endometrium or the decidua for N-glycosidic and O-glycosidic glycoprotein side chain production (N-glycan and O-glycan) of αCG and βhCG.
Continuation of procedure selection, establishing a master cell bank, working cell bank, culture expansion, fine purification, and quality control as in Example 1.

### EXAMPLE 3

### Treatment of Fertility Disorders/Treatment of Implant Disorders/Treatment of Early Pregnancy Losses

For the treatment of fertility and implantation disorders as well as for the treatment of early pregnancy losses the patient receives the hCG (comprised of α-CG and β6/7 hCG) produced as disclosed in connection with Example 1 or Example 2 on cycle day 21 and again every three days, 250 μg subcutaneously, up to the diagnosis of pregnancy.

### EXAMPLE 4

### Treatment of Miscarriages

In patients with imminent early or late miscarriage the patients receive by subcutaneous injection hCG (comprised of α-CG and β6/7 hCG) produced as described in Example 1 or Example 2 at a dosage of 250 μg α and β6/7 hCG twice per week. Beginning with the 20th week of gestation additionally every week up to the 28th week of gestation a dose of 1,000 μg of hCG (comprised of α-CG and β6/7 hCG) produced as described in Example 1 is instilled into the amniotic fluid.

### EXAMPLE 5

### Treatment of Premature Birth

In case of diagnosed imminent premature birth, the patients receive 1,000 μg of hCG (comprised of α-CG and β6/7 hCG) produced as described in Example 1 or Example 2 into the amniotic fluid. This instillation is repeated weekly up to the 32nd week of pregnancy. In addition, the patients are injected subcutaneously every day with 250 μg of α and β6/7 hCG.

### EXAMPLE 6

### Treatment of Preeclampsia

In case of severe preeclampsia the patients receive 500 μg of hCG (comprised of α-CG and β6/7 hCG) produced as described in Example 1 or Example 2 once a week instilled into the amniotic fluid. In addition, in intervals of three days 250 μg are injected subcutaneously.

### EXAMPLE 7

### Treatment of Growth Retardation

For treatment of growth retardation patients are treated up to the 34th week of gestation every other day with 250 μg of hCG (comprised of α-CG and β6/7 hCG) produced as described in Example 1 or Example 2 by subcutaneous injection.

### EXAMPLE 8

### Treatment of Autoimmune Diseases and for Induction of Immunological Tolerance in Transplant Patients

For treatment of autoimmune diseases 0.5 μg of the hCG (comprised of α-CG and β6/7 hCG) produced as described in Example 1 or Example 2 is prescribed to be taken sublingually three times a day.

For induction of immunological tolerance in case of transplant patients, the patients are subcutaneously injected every day with 50 μg of hCG (comprised of α-CG and β6/7 hCG) produced as described in Example 1 or Example 2. In addition, the patients, already before transplantation and 12 weeks subsequent thereto, are treated by intravenous application with mononuclear blood cells removed weekly from the patient and incubated in vitro for 8 hours with approximately 3 μg of hCG (comprised of α-CG and β6/7 hCG) produced as described in Example 1 or Example 2. In order to enable immune tolerance for transplanted organs, this organ must be provided with an immune-privilege space in that the organs are enclosed in a tightly fitting biomembrane from which every day 1 μg of hCG (comprised of α-CG and β6/7 hCG) produced as described in Example 1 or Example 2 is slowly released.

### EXAMPLE 9

### Preventing Graft-Versus-Host Reaction

For preventing a graft-versus-host reaction the prepared graft after removal from the donor is to be flushed with hCG (comprised of α-CG and β6/7 hCG) produced as described in Example 1 or Example 2 in a dosage of 250 μg/0.5 ml through the arteries as well as the veins. This hCG form is also added to the transport medium in the same concentration.

### EMBODIMENT 10

### Treatment of Sepsis

For treatment of sepsis daily 500 to 1,000 μg of the hCG (comprised of α-CG and β6/7 hCG) produced as described in Example 1 or Example 2 are infused. Virus-caused carcinoma and sarcoma are systemically and locally treated with doses of 1,000 μg/d.

### EMBODIMENT 11

### Use for Contraception

In this connection, daily 10 μg of the hCG (comprised of α-CG and 6/7 hCG) produced as described in Example 1 or Example 2 are subcutaneously injected or applied sublingually. Moreover, rods of polyethylene-co-vinyl acetate can be inserted for subcutaneous application or rings of polyethylene-co-vinyl acetate for intravaginal application that release every day 5 μg of hCG (comprised of α-CG and β6/7 hCG) produced as described in Example 1 or Example 2.

### EXAMPLE 12

### Use for Prophylaxis of HIV Infection

hCG-containing gels and creams that contain hCG (comprised of α-CG and β6/7 hCG) produced as described in Example 1 or Example 2 in a concentration of 1% are used for prophylaxis of HIV infection.

### EXAMPLE 13

### Treatment of Tissue Ischemia and Severe Necrosis

For treatment of severe tissue ischemia such as in case of apoplexia, heart attack, or severe postpartum brain edema in newborns every day a dosage of 500 to 1,000 μg of hCG (comprised of α-CG and β6/7 hCG) produced as described in Example 1 or Example 2 is infused or, in case of burns, applied locally in the form of sprays.

### EXAMPLE 14

### Treatment of Allergic Inflammatory Reactions

For treatment and prophylaxis of an allergic inflammatory reactions of the upper air passages (hay fever, bronchial asthma) 50 to 100 μg of hCG (comprised of α-CG and β6/7 hCG) produced as described in Example 1 or Example 2 is used in the form of a spray (alternatively a cream or gel).

### EXAMPLE 15

### Treatment of Benign Prostate Hyperplasia (BPH)

For treatment of the benign prostate hyperplasia (BPH) 0.5 μg of hCG (comprised of α-CG and β6/7 hCG) produced as described in Example 1 or Example 2 are administered sublingually twice a day.

### EXAMPLE 16

### Treatment of Autoimmune Disease of the Eye

In autoimmune uveitis in intervals of 4 to 7 days 0.5 ml of a solution is injected that contains 10 μg/ml of the hCG (comprised of α-CG and β6/7 hCG) produced as described in Example 1 or Example 2. This therapy can be prescribed also for a therapy-resistant glaucoma therapy or in case of risk of rejection of a cornea transplant.

### EXAMPLE 17

### Treatment of Multiple Sclerosis

In patients with multiple sclerosis weekly 2 ml hCG solution containing hCG (comprised of α-CG and β6/7 hCG) produced as described in Example 1 or Example 2 in a concentration of 10 μg/ml is instilled intrathecally.

### EXAMPLE 18

### Treatment of Crohn's Disease and Colitis Ulcerosa

Oral administration of hCG, comprised of α-CG and β6/7 hCG and produced as described in Example 1 or Example 2, in a (biomembrane) capsule that releases hCG in a concentration of 5 μg/ml only once it reaches the intestine or colon.

### EXAMPLE 19

### Transplantation of Autologous and Xenogenic Islet Cells

Autologous islet cells are stimulated before transplantation for 48 hours in an emulsion with 2 μg/ml of hCG (comprised of α-CG and β6/7 hCG) produced as described in Example 1 or Example 2 and subsequently injected intravenously.

Alternative: encapsulation of autologous and xenogenic islet cells in hCG-releasing (comprised of α-CG and β6/7-hCG) biomembranes comprised of biodegradable poly(ε-caprolactone) (PCL).

### EXAMPLE 20

### Treatment of Interstitial Cystitis and Chronic Cystitis

Insertion of a biodegradable implant of poly(ε-caprolactone) (PCL) in the form of a rod in case of chronic bladder inflammation or an interstitial cystitis which rod releases continuously hCG (produced as described in Example 1 or Example 2; comprised of α-CG and β6/7 hCG).

### EXAMPLE 21

### Treatment of HIV infection

In case of pronounced T-cell drop in connection with HIV infection 1,000 μg/ml of hCG (produced as described in Example 1 or Example 2; comprised of α-CG and β6/7 hCG) is intravenously injected daily for two weeks.

### EXAMPLE 22

Gene-technological production of recombinant αβhCG (βhCG gene β7-specific or gene β6-specific or gene β5-specific) in human epithelium cells of the secretory endometrium or the decidua with additional insertion of synthesis function of human N-glycosidic and O-glycosidic glycan substitution of the αCG and βhCG subunits in addition to insertion of βhCG and αCG as in Example 2.

In addition to the vectors named in Example 2 the human epithelium cells are co-transfected with a vector that contains proteins that are important for the human N-glycosidic and O-glycosidic glycoprotein side chain production.

Otherwise the same procedure as Example 2 is followed.

### EXAMPLE 23

Production of human native αβhCG with native N-glycosidic and O-glycosidic glycan side chain formation in physiological epithelium cells of the secretory transformation endometrium with selected βhCG β6 or βhCG β7 gene expression.
Isolation of endometrial or decidual luminal or gland epithelium after collagenase/DNAse cell dispersion and cell separation
Primary cell culture under optimal conditions (estradiol, progesterone, and other mediators) and selection of cells after sequence analysis, βhCG β6 or β7.
Continuation of procedure selection, establishing master cell bank, working cell bank, culture expansion, fine purification, and quality control as described in Example 1.

### EXAMPLE 24

In up to now unpublished primary cell culture examinations it has been found at the transcription as well as translation level that in the endometrium cell culture the formation of βhCG and αCG subunits can be induced by mediators such as estradiol, progesterone, hCG, Th2-cytokine, and LPS and reduced by inhibitors.

### EXAMPLE 25

In up to now unpublished primary cell culture examples it has been found at the transcription as well as translation level that in the endometrium cell culture the formation of βhCG and αCG subunits can be induced by mediators such as estradiol, progesterone, hCG, Th2-cytokine, and LPS and reduced by inhibitors.

### CITED NON-PATENT LITERATURE

(1) J. C. Pierce, T. F. Parsons, Annu. Rev. Biochem., 50 (1981) 465-495
(2) E. Reshef, Z. M. Lei, C. V. Rao et al., J. Clin. Endocrinol. Metab., 70 (1990) 421-430
(3) P. Licht, H. Cao, Z. M. Lei et al., Endocrinology, 133 (1993) 3014-3025
(4) J. Lin, S. Lojun, Z. M. Lei et al., Mol. Cell Endocrinol., 111 (1995) 13-17
(5) J. M. Zhang, Ch. V. Rao, Z. M. Lei et al., Am. J. Reprod. Immunol., 49 (2003) 93-100
(6) P. Licht M. v. Wolff, A. Berklitz et al., Fertil. Steril., 79 (2003) 718-723
(7) R. I. Cruz, D. M. Anderson, E. G. Armstrong et al., J. Clin. Endocrinol. Metab., 64 (1987) 433-440
(8) J. L. Jameson, and A. N. Hollenberg, Endocrine Rev., 14 (1993) 203-221
(9) P. Policastro, C. Ovitt, M. Hoshinaet al., J. Biol. Chem., 258 (1983) 11492-11499
(10) K. Talmadge, N. C. Vamvakopoulus and J. C. Fiddes, Nature, 307 (1984) 37-40
(11) M. Bo and I. J. Boime, J. Biol. Chem., 267 (1992) 3179-3184
(12) J. F. Fiddes and H. M. Goodman, Nature, 281 (1979) 351-356
(13) P A Rothman, V. A. Chao, M. R. Taylor et al., Mol. Reprod. Dev., 33 (1992) 1-6
(14) S. Dirnhofer, M. Hermann, A. Hittmair et al., J. Clin. Endocrinol. Metab., 81 (1996) 4212-4217
(15) Z. M. Lei, P. Toth, C. V. Rao and D. Pridham, J. Clin. Endocrinol. Metab., 77 (1993) 863-972
(16) T. Yokotani, T. Koizumi, R. Taniguchi et al., IntJ. Cancer, 71 (1997) 539-544
(17) P. Berger, W. Kranewitter, S. Madersbacher et al., FEBS Lett., 343 (1994) 229-233
(18) H. Alexander, G. Zimmermann et al., Hum. Reprod. Update, 4 (1998) 550-559
(19) G. W. Wolkersdorfer, S. R. Bomstein, G. Zimmermann et al., Mol. Hum. Reprod., 4 (1998)179-184
(20) G. Zimmermann, D. Baier, J. Majer and H. Alexander, Mol. Hum. Reprod., 9 (2003) 81-89
(21) D. Bellet, V. Lazar, I. Bieche et al., Cancer Res., 57 (1997) 516-523
(22) C. F. Barker and R E Billingham, Adv. Immunol., 25 (1977) 1-54
(23) B. R. Ksander and J. W. Streilein, Chem. Immunol., 58 (1994) 117-145
(24) R. A. Pattillo, M. R. Shalaby, R. O. Hussa et al., Obstet. Gynecoi, Al (1976) 557-561
(25) B. Nisula and A. Bartocci, Ann. d'Endocrinol., 45 (1984) 315-319
(26) U. C. Hedge, Med. Hypotheses, 35 (1991) 159-164
(27) J. L. Bartha, R. Romero-Carmona, M. Escobar-Llompart et al., Obstet. Gynecoi., 102, 995-999
(28) P. Toth, Semin. Reprod. Med., 19, 55-61
(29) J. Zaidi, R. Pittrof, A. Shaker et al., Fertil. Steril., 65 (1996) 377-381
(30) A J. Ziecik, G. Golba and J. Kiesielewska, Exp. Ciln. Endocrinol. Diabetes, 104 (1996)158-163
(31) L.-H. Garn and A. Latiff, Int. J. Biol. Sei., 1 (2005) 103-109
(32) Garcia-Campayo, V. et al., 194 (2002) 63-70
(33) F. Fandrich, X. Lin et al., Transplant. Proc, 30 (1998) 2360-2361
(34) F. Fandrich, X. Lin et al., Nat. Med., 8 (2002) 171-178
(35) M. M. Matzuk, J. L. Keene et al. J. Biol. Chem., 264 (1989) 2409-2414
(β6) V. Garcia-Campayo, T. Sugahara, et al., Mol. Cell Endocrinol. 194 (2002) 63-70
(37) S . Birken, P. Berger et al., Clin. Chem., 49 (2003) 144-154
(38) C M. Howles, Hum. Reprod. Update, 2 (1996) 172-191

What is claimed is:

1. Medicament, in particular for treating pregnancy disorders or for inducing an immunological tolerance in patients with autoimmune diseases or transplantation processes, comprising at least one each of:
a) a precursor hCG β subunit of the human choriongonadotropine selected from hCG β6 according to SEQ ID NO 1 or SEQ ID NO 2 and hCG β7 according to SEQ ID NO 5 or a mature hCG β subunit selected from hCG β6 according to SEQ ID NO 3 or SEQ ID NO 4 and hCG β7 according to SEQ ID NO 6 or glycolised fragments of these sequences;
b) a precursor α subunit of the human choriongonadotropine according to
SEQ ID NO 9 or the mature α subunit of the human choriongonadotropine according to SEQ ID NO 10 or glycolysed fragments of these sequences,
wherein the β subunits and the α subunits are preferably used in equimolar quantities.

2. Medicament according to claim 1, wherein:
a) the precursor hCG β subunit β6 according to SEQ ID NO 1 or SEQ ID NO 2 or β7 according to SEQ ID NO 5 is glycolysed at least at one of the following amino acids: Asn-33, Asn-50, Ser-141, Ser-147, Ser-152, Ser-158 and/or
b) the mature β subunit β6 according to SEQ ID NO 3 or SEQ ID NO 4 or hCG β7 according to SEQ ID NO 6 is glycolysed at least at one of the following amino acids: Asn-13, Asn-30, Ser-121, Ser-127, Ser-132, Ser-138 and/or
c) the precursor-hCG α subunit according to SEQ ID NO 9 is glycolysed at least at one of the following amino acids: Asn-76, Asn-102 and/or
d) the mature α subunit according to SEQ ID NO 10 is glycolysed at least at one of the following amino acids: Asn-52, Asn-78.

3. Medicament according to claim 1, wherein the precursor hCG β subunit, the mature hCG β subunit, the precursor α subunit, the mature α subunit and/or the fragments are recombinant-produced.

4. Medicament according to claim 1, wherein the medicament is prepared for parenteral administration or a subcutaneous injection.

5. Medicament according to claim 1, wherein the medicament is prepared such that the quantity of administered human choriongonadotropine is 3 to 6 μg per kg body weight per day.

6. Method for treating pregnancy disorders or for inducing an immunological tolerance in patients with autoimmune diseases or transplantation processes, comprising the steps of:
1) combining a precursor hCG β subunit of the human choriongonadotropine selected from hCG β6 according to SEQ ID NO 1 or SEQ ID NO 2 and hCG β7 according to SEQ ID NO 5 or a mature hCG β subunit selected from hCG β6 according to SEQ ID NO 3 or SEQ ID NO 4 and hCG β7 according to SEQ ID NO 6 or glycolised fragments of these sequences with a precursor α subunit of the human choriongonadotropine according to SEQ ID NO 9 or the mature α subunit of the human choriongonadotropine according to SEQ ID NO 10 or glycolysed fragments of these sequences;
2) administering the composition of step 1) in an effective quantity to a patient;
wherein the β subunits and the α subunits are preferably used in equimolar quantities.

7. Method according to claim 6, wherein:
a) the precursor hCG β subunit β6 according to SEQ ID NO 1 or SEQ ID NO 2 or β7 according to SEQ ID NO 5 is glycolysed at least at one of the following amino acids: Asn-33, Asn-50, Ser-141, Ser-147, Ser-152, Ser-158 and/or
b) the mature β subunit β6 according to SEQ ID NO 3 or SEQ ID NO 4 or hCG β7 according to SEQ ID NO 6 is glycolysed at least at one of the following amino acids: Asn-13, Asn-30, Ser-121, Ser-127, Ser-132, Ser-138 and/or
c) the precursor-hCG α subunit according to SEQ ID NO 9 is glycolysed at least at one of the following amino acids: Asn-76, Asn-102 and/or
d) the mature α subunit according to SEQ ID NO 10 is glycolysed at least at one of the following amino acids: Asn-52, Asn-78.

8. Method according to claim 6, wherein the precursor hCG β subunit, the mature hCG β subunit, the precursor α subunit, the mature α subunit and/or the fragments are recombinant-produced.

9. (canceled)

10. Method according to claim 6, wherein the pregnancy disorder is a fertility disorder, an implantation disorder, early pregnancy loss, imminent and habitual abortion as well as premature birth, growth retardation or preeclampsia.

11. Method according to claim 6, wherein in step 2) the composition is administered parenterally or by subcutaneous or intravenous injection.

12. Method according to claim 6, wherein 3 to 6 μg of human choriongonadotropine per kg body weight per day are administered.

13. Method according to claim 6, wherein mononuclear blood cells removed from the patient are treated in vitro with the composition of step 1) and are reinjected subcutaneously or intravenously into the patient


SEQUENCE LISTING (25 sequences)

SEQ ID NO: 1 - protein, 165 aa, Homo sapiens [sequence omitted]

SEQ ID NO: 2 - protein, 165 aa, Homo sapiens
  hCG beta6b, precursor, human endometrium
MEMFQGLLLLLLLSMGGTWASREPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQASSSSKAPPPSLPSPSRLPGPSDTPILPQ

SEQ ID NO: 3 - protein, 145 aa, Homo sapiens
  hCG beta6a, mature form, human endometrium
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQASSSSKAPPPSLPSPSRLPGPSDTPILPQ

SEQ ID NO: 4 - protein, 145 aa, Homo sapiens
  hCG beta6b, mature form, human endometrium
SREPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQASSSSKAPPPSLPSPSRLPGPSDTPILPQ

SEQ ID NO: 5 - protein, 165 aa, Homo sapiens
  hCG beta7, precursor, human endometrium
MEMFQGLLLLLLLSMGGTWASREMLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQASSSSKAPPPSLPSPSRLPGPSDTPILPQ

SEQ ID NO: 6 - protein, 145 aa, Homo sapiens
  hCG beta7, mature form, human endometrium
SREMLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQASSSSKAPPPSLPSPSRLPGPSDTPILPQ

SEQ ID NO: 7 - protein, 165 aa, Homo sapiens
  hCG beta5, beta8, beta3, precursor, human throphoblast
MEMFQGLLLLLLLSMGGTWASKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 8 - protein, 145 aa, Homo sapiens
  hCG beta5, beta8, beta3, mature form, human throphoblast
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 9 - protein, 116 aa, Homo sapiens
  hCG alpha, precursor
MDYYRKYAAIFLVTLSVFLHVLHSAPDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 10 - protein, 92 aa, Homo sapiens
  hCG alpha, mature form
APDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 11 - DNA, 861 bp, Homo sapiens
  hCG beta 6a cDNA (1-377  exon 1, 378-545 exon 2, 546-861  exon 3)
agcactttcctcgggtcacggcctcctcctggttcccaagaccccaccataggcagaggcaggccttcctacaccctactctctgtgcctccagcctcgactagtccctaacactcgacgactgagtctcagaggtcacttcaccgtggtctccgcctcatccttggcgctagaccactgaggggagaggactggggtgctccgctgagccactcctgtgcctccctggccttgtctacttctcgccccccgaagggttagtgtcgagctcactccagcatcctacaacctcctggtggccttgccgcccccacaaccccgaggtatgaagccaggtacaccaggcaggggacgcaccaaggatggagatgttccaggggctgctgctgttgctgctgctgagcatgggcgggacatgggcatccaaggagccgcttcggccacggtgccgccccatcaatgccaccctggctgtggagaaggagggctgccccgtgtgcatcaccgtcaacaccaccatctgtgccggctactgccccaccatgacccgcgtgctgcagggggtcctgccggccctgcctcaggtggtgtgcaactaccgcgatgtgcgcttcgagtccatccggctccctggctgcccgcgcggcgtgaaccccgtggtctcctacgccgtggctctcagctgtcaatgtgcactctgccgccgcagcaccactgactgcgggggtcccaaggaccaccccttgacctgtgatgacccccgcttccaggcctcctcttcctcaaaggcccctccccccagccttccaagtccatcccgactcccggggccctcggacaccccgatcctcccacaataaa

SEQ ID NO: 12 - DNA, 861 bp, Homo sapiens
  hCG beta 6b cDNA (1-377  exon 1, 378-545 exon 2, 546-861  exon 3)
agcactttcctcgggtcacggcctcctcctggttcccaagaccccaccataggcagaggcaggccttcctacaccctactctctgtgcctccagcctcgactagtccctaacactcgacgactgagtctcagaggtcacttcaccgtggtctccgcctcatccttggcgctagaccactgaggggagaggactggggtgctccgctgagccactcctgtgcctccctggccttgtctacttctcgccccccgaagggttagtgtcgagctcactccagcatcctacaacctcctggtggccttgccgcccccacaaccccgaggtatgaagccaggtacaccaggcaggggacgcaccaaggatggagatgttccaggggctgctgctgttgctgctgctgagcatgggcgggacatgggcatccagggagccgcttcggccacggtgccgccccatcaatgccaccctggctgtggagaaggagggctgccccgtgtgcatcaccgtcaacaccaccatctgtgccggctactgccccaccatgacccgcgtgctgcagggggtcctgccggccctgcctcaggtggtgtgcaactaccgcgatgtgcgcttcgagtccatccggctccctggctgcccgcgcggcgtgaaccccgtggtctcctacgccgtggctctcagctgtcaatgtgcactctgccgccgcagcaccactgactgcgggggtcccaaggaccaccccttgacctgtgatgacccccgcttccaggcctcctcttcctcaaaggcccctccccccagccttccaagtccatcccgactcccggggccctcggacaccccgatcctcccacaataaa

SEQ ID NO: 13 - DNA, 861 bp, Homo sapiens
  hCG beta 7 cDNA (1-377  exon 1, 378-545 exon 2, 546-861  exon 3)
agcacttttctcgggtcacggcctcctcctggttcccaagaccccaccataggcagaggcaggccttcctacaccctactctctgtgcctccagcctcgactagtccctagcactcgacgactgagtctcagaggtcacttcaccgtggtctccgcctcatccttggtgctagaccactgaggggagaggactggggtgctccgctgagccactcctgtgcctccctggccttgtctacttctcgccccccgaagggttagtgtccagctcactccagcatcctacaacctcctggtggccttgacgcccccacaaacccgaggtataaagccaggtacaccaggcaggggacgcaccaaggatggagatgttccaggggctgctgctgttgctgctgctgagcatgggcgggacatgggcatccagggagatgcttcggccacggtgccgccccatcaatgccaccctggctgtggagaaggagggctgccccgtgtgcatcaccgtcaacaccaccatctgtgccggctactgccccaccatgacccgcgtgctgcagggggtcctgccggccctgcctcaggtggtgtgcaactaccgcgatgtgcgcttcgagtccatccggctccctggctgcccgcgcggcgtgaaccccgtggtctcctacgccgtggctctcagctgtcaatgtgcactctgccgccgcagcaccactgactgcgggggtcccaaggaccaccccttgacctgtgatgacccccgcttccaggcctcctcttcctcaaaggcccctccccccagccttccaagtccatcccgactcccggggccctcggacaccccgatcctcccacaataaa

SEQ ID NO: 14 - DNA, 861 bp, Homo sapiens
  hCG beta 5 cDNA (1-377  exon 1, 378-545 exon 2, 546-861  exon 3)
agcactttgctcgggtcacggcctcctcctggctcccaggaccccaccataggcagaggcaggccttcctacaccctactccctgtgcctccaggctcgactagtccctagcactcgacgactgagtctctgagatcacttcaccgtggtctccgcctcacccttggtgctggaccagtgagaggagagggctggggcgctccgctgagccactcctgcgcccccctggccttgtctacctcttgccccccgaagggttagtgtcgagctcaccccagcatcctacaacctcctggtggccttgccgcccccacaaccccgaggtataaagccaggtacacgaggcaggggacgcaccaaggatggagatgttccaggggctgctgctgttgctgctgctgagcatgggcgggacatgggcatccaaggagccgcttcggccacggtgccgccccatcaatgccaccctggctgtggagaaggagggctgccccgtgtgcatcaccgtcaacaccaccatctgtgccggctactgccccaccatgacccgcgtgctgcagggggtcctgccggccctgcctcaggtggtgtgcaactaccgcgatgtgcgcttcgagtccatccggctccctggctgcccgcgcggcgtgaaccccgtggtctcctacgccgtggctctcagctgtcaatgtgcactctgccgccgcagcaccactgactgcgggggtcccaaggaccaccccttgacctgtgatgacccccgcttccaggactcctcttcctcaaaggcccctccccccagccttccaagtccatcccgactcccggggccctcggacaccccgatcctcccacaataaa

SEQ ID NO: 15 - DNA, 348 bp, Homo sapiens
  hCG alpha cDNA (1-348 encoding precursor, 73-348 encoding mature form of protein)
atggattactacagaaaatatgcagctatctttctggtcacattgtcggtgtttctgcatgttctccattccgctcctgatgtgcaggattgcccagaatgcacgctacaggaaaacccattcttctcccagccgggtgccccaatacttcagtgcatgggctgctgcttctctagagcatatcccactccactaaggtccaagaagacgatgttggtccaaaagaacgtcacctcagagtccacttgctgtgtagctaaatcatataacagggtcacagtaatggggggtttcaaagtggagaaccacacggcgtgccactgcagtacttgttattatcacaaatct

SEQ ID NO: 16 - DNA, 17 bp, artificial
  primer 1, sense beta-hCG
tcgggtcacggcctcct

SEQ ID NO: 17 - DNA, 20 bp, artificial
  primer 2, sense beta-hCG
tcacttcaccgtggtctccg

SEQ ID NO: 18 - DNA, 20 bp, artificial
  primer 3, sense beta-hCG
ggctgtggagaaggagggct

SEQ ID NO: 19 - DNA, 18 bp, artificial
  primer 4, anti-sense beta-hCG
cagcacgcgggtcatggt

SEQ ID NO: 20 - DNA, 23 bp, artificial
  primer 5, anti-sense beta-hCG
ggaagcgggggtcatcacaggtc

SEQ ID NO: 21 - DNA, 17 bp, artificial
  primer 6, anti-sense beta-hCG
tcggggtgtccgagggc

SEQ ID NO: 22 - DNA, 20 bp, artificial
  primer 7, sense alpha-hCG
tgcaggattgcccagaatgc

SEQ ID NO: 23 - DNA, 20 bp, artificial
  primer 8, anti-sense alpha-hCG
ccgtgtggttctccactttg

SEQ ID NO: 24 - DNA, 18 bp, artificial
  primer 9, sense GAPDH
ccatggagaaggctgggg

SEQ ID NO: 25 - DNA, 21 bp, artificial
  primer 10, anti-sense GAPDH
ccaaagttgtcatggatgacc